(19) United States
(12) Patent Application Publication
(10) Pub. No.: US 2012/0015031 A1
(43) Pub. Date: Jan. 19, 2012

(54) NOVEL GASTRO-RETENTIVE DOSAGE FORMS

(75) Inventor: Ramesh SESHA, Windsor, NJ (US)

(73) Assignee: Grunenthal GmbH, Aachen (DE)

(21) Appl. No.: 13/181,605

(22) Filed: Jul. 13, 2011

Related U.S. Application Data
(60) Provisional application No. 61/399,589, filed on Jul. 14, 2010.

(51) Int. Cl. A61K 9/24; A61P 25/04; A61K 31/485; A61P 25/00; A61K 31/167; A61K 9/14

(52) U.S. Cl. 424/472; 514/630; 514/282

(57) ABSTRACT

A gastro-retentive pharmaceutical dosage form comprising a therapeutically effective amount of at least one opioid, at least one form of acetaminophen, optionally an opioid antagonist and at least one pharmaceutically acceptable excipient, which dosage form is retained in the stomach for at least four hours and is suitable for once daily or twice daily administration. Also provided is a method of treating pain by administering the dosage form to a patient in need thereof. The acetaminophen is either in slow release form or in immediate release form or as combination of both.

### PRIORITY CLAIM

This application claims priority of U.S. Provisional Application Ser. No. U.S. 61/399,589 filed on Jul. 14, 2010, the entire contents of which are incorporated herein by reference.

### FIELD OF INVENTION

The present invention is related to gastro-retentive pharmaceutical dosage forms comprising an opioid and acetaminophen that are retained in stomach for at least four hours and are suitable for twice daily or once daily administration to treat a disorder in mammal and the methods of using such dosage forms.

### BACKGROUND OF INVENTION

Opioid or opioid agonists class of drugs include morphine, the archetypical opioid, and various others such as, for example, codeine, dihydrocodeine, hydrocodone, hydromorphone, levorphanol, meperidine, buprenorphine, fentanyl, fentanyl derivatives, dipipanone, heroin, tapentadol tramadol, etorphine, dihydroetorphine, butorphanol, methadone, diamorphine, oxycodone, oxymorphone, pethidine and propoxyphene, etc. Opioid agonists chemically interact with areas or binding sites of the central nervous system related to the perception of pain, to movement, mood and behavior, and to the regulation of neuroendocrinological functions. Opioid agonists exhibit pharmacological properties that provide a range of therapeutic uses for patients in addition to analgesic use. Opioid agonists have been prescribed for effective use as hypnotics, sedatives, anti-diarrheal, anti-spasmodic, and anti-tussives.

Analgesic drugs particularly Opioids are often administered in combination with other analgesics such as acetaminophen. However, almost every combination drug approved is immediate release formulation that require medication every 4 to six hours to efficiently relive pain. For Example, ULTRACET® (tramadol hydrochloride/acetaminophen) Tablets combines two analgesics, tramadol 37.5 mg and acetaminophen 325 mg, similarly Percocet® combines acetaminophen with oxycodone, Vicodin® is marketed as combination of acetaminophen with hydrocodone. Currently, extended-release oral dosage forms are all for delivering a single active pharmaceutical ingredient-Ultram®, Oxycontin®, Tylenol® etc to provide controlled release of oxycodone hydrochloride via twice-daily administration.

Thus, there is a strong unmet medical need for long acting fixed dose combination drugs of an opioid and acetaminophen. Considering that such drugs are often used over a long term by elderly patients to manage pain that is often chronic, compositions that can help reduce the dosage or frequency of either or both of drug types without comprising the therapeutic benefits would fill this medical unmet need.

Opioids have been combined with other drugs including non-opioid analgesic agents, to try to lower the amount of opioid needed to produce an equivalent degree of analgesia and reduce the side effects from opioids. It has been reported that some of these combination products also have a synergistic analgesic effect. U.S. Pat. No. 4,571,400 discloses a combination of dihydrocodeine, an opioid analgesic, and ibuprofen, a non-opioid analgesic. See also U.S. Pat. Nos. 4,587,252 and 4,569,937, which disclose other ibuprofen opioid combinations. Combinations of non-opioid analgesics have also been prepared to avoid the side effects associated with opioids, and the combinations are noted to have the benefit of requiring less of each ingredient and may provide additive effects, U.S. Pat. No. 4,260,629, U.S. Pat. No. 4,132,788. A number of sustained release formulations for the delivery of a combination of acetaminophen and tramadol have been described, for example, in U.S. Pat. No. 7,374,781 and U.S. Patent Publication No. US2003/0092724 A1. U.S. Pat. No. 6,558,701 describes a combination of tramadol and diclofenac. U.S. Pat. No. 20090130183 describes a sustained release formation of tramadol and acetaminophen and U.S. Pat. No. 20100015222 discloses a dosage form of acetaminophen and an opioid for delivery of the drugs to the upper gastrointestinal tract (“GI”) of a mammal for an extended period of time.

However, there is no approved gastro-retentive combination of opioid and acetaminophen nor do the prior art disclose a gastro-retentive dosage form that is retained in the stomach for at least four hours and is suitable for once or twice daily administration. Further prior art doesn't disclose a method of treating pain or pain related disorder wherein the method comprises administering to a mammal in need thereof, a dosage form comprising a therapeutically effective amount of an opioid, at least one form of acetaminophen and at least one pharmaceutically acceptable excipient, wherein the said dosage form is retained in the stomach for at least four hours and is suitable for twice daily and once daily administration. Such dosage forms are useful because Acetaminophen is generally absorbed quickly, produces a quick onset of analgesia but has a short half-life leading to short duration. Opioids on the other hand have a delayed onset but longer duration compared to acetaminophen.

The present inventor while working on the analgesic combinations have surprisingly found dosage forms and a method of treating moderate to severe painful conditions, by administering to a subject in need thereof, a gastro-retentive dosage form comprising an opioid, at least one form of acetaminophen and at least one pharmaceutically acceptable excipient wherein the dosage form is retained in the stomach for at least four hours and is administered once daily or twice daily administration so as to provide better pain management.

Towards achieving this objective, this invention discloses a dosage form comprising a therapeutically effective amount of an opioid, at least one form of acetaminophen and at least one pharmaceutically acceptable excipient, wherein the said dosage form is retained in the stomach for at least four hours and is suitable for twice daily and once daily administration. The invention further discloses a method of treating pain and pain related disorder in a mammal comprising administering to patient in need thereof, a dosage form comprising a therapeutically effective amount of a slow release tapentadol, at least one form of acetaminophen and at least one pharmaceutically acceptable excipient, wherein the said dosage form is retained in the stomach for at least four hours and is suitable for twice daily and once daily administration. This invention is advantageous as there will be a decreased dosing of the active ingredient, with substantial patient compliance and sustained period of pain relief. The invention further discloses a dosage form comprising an opioid, at least form of acetaminophen and at least one opioid antagonist wherein the dosage form is suitable for once daily or twice daily administration.

### BRIEF DESCRIPTION OF THE INVENTION

The present invention provides a pharmaceutical dosage form comprising a therapeutically effective amount of at least one opioid, at least one form of acetaminophen and at least one pharmaceutically acceptable excipient wherein the said dosage form is retained in the stomach for at least four hours and is suitable for once daily or twice daily administration.

The present invention further provides a method of treating a disorder by administering a pharmaceutical dosage form comprising a therapeutically effective amount of at least one opioid, at least one form of acetaminophen and at least one pharmaceutically acceptable excipient, wherein the said dosage form is retained in the stomach for at least four hours and is suitable for once daily or twice daily administration.

The invention further provides a pharmaceutical dosage form comprising a therapeutically effective amount of at least one opioid, at least one form of acetaminophen and at least one pharmaceutically acceptable excipient, wherein the said acetaminophen is in immediate release form and the dosage form is retained in the stomach for at least four hours and is suitable for once daily or twice daily administration.

The invention further provides a pharmaceutical dosage form comprising a therapeutically effective amount of at least one opioid, at least one form of acetaminophen and at least one pharmaceutically acceptable excipient, wherein acetaminophen is in slow release form and the dosage form is retained in the stomach for at least four hours and is suitable for once daily or twice daily administration.

The invention further provides a pharmaceutical dosage form comprising a therapeutically effective amount of at least one opioid, at least one form of acetaminophen and at least one pharmaceutically acceptable excipient, wherein acetaminophen is in partially in immediate release form and partially in slow release form and the dosage form is retained in the stomach for at least four hours and is suitable for once daily or twice daily administration.

The present invention further provides a method of treating a disorder by administering a pharmaceutical dosage form comprising a therapeutically effective amount of at least one opioid, at least one form of acetaminophen and at least one pharmaceutically acceptable excipient, wherein the said dosage form is retained in the stomach for at least four hours and is suitable for once daily or twice daily administration.

The present invention further provides a method of treating a disorder by administering a pharmaceutical dosage form comprising a therapeutically effective amount of at least one opioid, at least one form of acetaminophen and at least one pharmaceutically acceptable excipient, wherein both acetaminophen and opioid are in slow release forms and the dosage form is retained in the stomach for at least four hours and is suitable for once daily or twice daily administration.

The present invention further provides a pharmaceutical dosage form comprising: a) a core comprising at least two release layers wherein a first release layer comprising at least one opioid with or without acetaminophen, and a second release layer comprises acetaminophen, with or without an opioid and at least one pharmaceutically acceptable excipient, b) a coat comprising the said core, wherein the dosage form is retained in the stomach for at least four hours and is suitable for once daily or twice daily administration.

The present invention further provides a method of treating a disorder by administering a pharmaceutical a dosage form comprising: a) a core comprising at least two release layers wherein a first release layer comprising at least one opioid with or without acetaminophen, and a second release layer comprises acetaminophen, with or without an opioid and at least one pharmaceutically acceptable excipient, b) a coat comprising the said core, wherein the dosage form is retained in the stomach for at least four hours and is suitable for once daily or twice daily administration.

The present invention further provides a pharmaceutical a dosage form comprising a) a compressed core comprising at least two release layers wherein a first release layer comprising at least one opioid, with or without acetaminophen, dispersed in a slow-release matrix; and a second release layer comprising acetaminophen, with or without an opioid, dispersed in a second release matrix; b) at least one permeable membrane covering comprising the said core; c) an encapsulating coat and the said dosage form is suitable for once daily or twice daily administration.

The present invention further provides a method of treating a disorder by administering a pharmaceutical a dosage form comprising a) a compressed core comprising at least two release layers wherein a first release layer comprising at least one opioid, with or without acetaminophen, dispersed in a slow-release matrix; and a second release layer comprising acetaminophen, with or without an opioid, dispersed in a second release matrix; b) at least one permeable membrane covering comprising the said core; c) an encapsulating coat and the said dosage form is suitable for once daily or twice daily administration.

The present invention further provides a pharmaceutical dosage form comprising a therapeutically effective amount of at least one opioid, at least one form of acetaminophen, and at least one pharmaceutically acceptable excipient, wherein the said dosage form is retained in the stomach for at least four hours and the said opioid is selected from a group consisting of alfentanil, axomadol, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, faxeladol, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papavereturn, pentazocine, phenadoxone, phenomorphan, phenazocine, phenoperidine, piminodine, piritramide, propheptazine, promedol, properidine, propoxyphene, sufentanil, tilidine, tapentadol, and, tramadol, and the said dosage form is suitable for once daily or twice daily administration.

The present invention further provides a method of treating pain by administering a pharmaceutical dosage form comprising a therapeutically effective amount of at least one opioid, at least one form of acetaminophen and at least one pharmaceutically acceptable excipient, wherein the said dosage form is retained in the stomach for at least four hours and the said opioid is selected from a group consisting of alfentanil, axomadol, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, faxeladol, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papavereturn, pentazocine, phenadoxone, phenomorphan, phenazocine, phenoperidine, piminodine, piritramide, propheptazine, promedol, properidine, propoxyphene, sufentanil, tilidine, tapentadol, and, tramadol, and the said dosage form is suitable for once daily administration and the said dosage form is suitable for once daily or twice daily administration.

The invention further provides a dosage form comprising an opioid, at least form of acetaminophen and at least one opioid antagonist wherein the dosage form is suitable for once daily or twice daily administration.

In one embodiment, the present invention provides a dosage form comprising at least one opioid, at least one form of acetaminophen with at least one pharmaceutically acceptable carrier, wherein the said dosage form exhibits a dissolution profile when tested in a USP type 1 apparatus at 100 rpm in 900 ml, pH 7.5 phosphate buffer and at 37′ C, such that:

after 2 hours from about 0 to about 65% of opioid is released;
after 4 hours from about 10% to about 85% of opioid is released;
after 8 hours from about 30 to about 90% of opioid is released;
after 12 hours more than 50% of opioid is released;
after 16 hours more than 60% of opioid is released; and after 20
hours more than 70% of opioid is released.

It is also on object of present invention to provide a dosage form comprising at least one opioid, at least one form of acetaminophen with at least one pharmaceutically acceptable carrier, wherein the dosage form upon initial administration of one dose, provides an early onset of clinical effect achieving a mean opioid plasma concentrations of at least 40 ng/mL from about 1 hour to at least 12 hours after administration and exhibits a dissolution profile, when tested in a USP type 1 apparatus at 100 rpm in 900 ml, pH 7.5 phosphate buffer and at 37′ C, such that:

after 2 hours from about 0 to about 75% of opioid is released;
after 4 hours from about 10% to about 85% of opioid is released;
after 8 hours from about 30 to about 90% of opioid is released;
after 12 hours more than 50% of opioid is released;
after 16 hours more than 60% of opioid is released; and after 20
hours more than 70% of opioid is released.

In another embodiment of the invention, the pharmaceutical composition for oral administration is in the form of a tablet or capsule comprising an opioid, at least one form of acetaminophen and at least pharmaceutical excipient. Preferably, the composition is in the form of a tablet.

In another embodiment of the invention, the pharmaceutical composition for oral administration is in the form of a tablet or capsule comprising an opioid, at least one form of acetaminophen and at least pharmaceutical excipient. Preferably, the composition is in the form of a capsule.

In at least one embodiment of the invention, a dosage form comprising at least one opioid, at least one form of acetaminophen with pharmaceutically acceptable carrier, wherein the dosage form is retained in the stomach for at least four hours and comprises from about 1 mg to about 1000 mg of opioid.

### BRIEF DESCRIPTION OF THE DRAWINGS

FIG. 1: The WOMAC Global Score from human clinical trial of Slow Release Tapentadol+Acetaminophen twice daily investigative drug against Slow Release Tapentadol two times daily and Acetaminophen two times daily reference drugs and Placebo.

FIG. 2: The WOMAC Subscale Stiffness Score from human clinical trial of Slow Release Tapentadol+Acetaminophen twice daily investigative drug against Slow Release Tapentadol two times daily and Acetaminophen two times daily reference drugs and Placebo.

FIG. 3: The WOMAC Subscale Physical Function Score from human clinical trial of Slow Release Tapentadol+Acetaminophen twice daily investigative drug against Slow Release Tapentadol two times daily and Acetaminophen two times daily reference drugs and Placebo.

FIG. 4: The WOMAC Subscale Pain Score from human clinical trial of Slow Release Tapentadol+Acetaminophen twice daily investigative drug against Slow Release Tapentadol two times daily and Acetaminophen two times daily reference drugs and Placebo.

### DETAILED DESCRIPTION OF THE INVENTION

The term “administration or ingestion” used herein means administration of dose of a formulation containing an active ingredient administered to a patient or subject.

The term “amylose” as used herein means a linear polymer of glucose and made of several thousand glucose units.

The term “binding agent” as used in this specification, refers to any conventionally known pharmaceutically acceptable binder such as polyvinyl pyrrolidone, hydroxypropyl cellulose, hydroxyethyl cellulose, hydroxypropyl methylcellulose, ethyl cellulose, polymethacrylate, polyvinyl alcohol, waxes and the like. Mixtures of the aforementioned binding agents may also be used. The preferred binding agents are water soluble materials such as polyvinyl pyrrolidone having a weight average molecular weight of 25,000 to 3,000,000. The binding agent may comprise approximately about 0 to about 40% of the total weight of the core and preferably about 3% to about 15% of the total weight of the core. In one embodiment, the use of a binding agent in the core is optional.

The term “clinical effect” as used herein as clinical efficacy with respect to pain experienced by study subjects measured using a suitable scale, for example; WOMAC global score, Likert-scale, or VAS score.

The term “controlled-release” as used herein is defined to mean a substantially gradual rate of release of the drug in the first once daily controlled-release dosage form or the at least one means for controllably releasing the in a substantially controlled manner per unit time in-vivo. The rate of release of the drug is controlled by features of the dosage form and/or in combination with physiologic or environmental conditions rather than by physiologic or environmental conditions alone.

The term “controlled-release dosage forms” or dosage forms which exhibit a “controlled-release” of acetaminophen or an Opioid as used herein is defined to mean dosage forms administered once daily that release drug at a relatively constant rate and provide plasma concentrations of the active drug that remain substantially invariant with time within the therapeutic range of the active drug over about a 24-hour period.

The term “cross linked amylose” as used herein means amylase units linked with one another.

The term “covering” as herein means a pharmaceutically acceptable retarding covering such as a film or a coating.

The term “candidate for sustained release” encompasses all the characteristics of a drug which make it a candidate for formulating it into an extended release fashion like a short elimination half life and consequent dosing of more than once a day, a single dose product given in an extended fashion to achieve better clinical results and avoid side effects associated with an immediate release etc.

The term “delayed-release dosage forms” or dosage forms which exhibit a “delayed-release” of the drug as used herein is defined to mean dosage forms administered once daily that do not substantially release drug immediately following administration but at a later time. Delayed-release dosage forms provide a time delay prior to commencement of drug-absorption. Such dosage forms will desirably be coated with a delayed-release coat.

The term “dosage form” as used herein is defined to mean a solid oral pharmaceutical preparation or system in which doses of medicine or active drug are included. A dosage form will desirably comprise, for example, at least one slow release dosage form including various slow release forms such as, osmosis controlled-release dosage form, erosion controlled-release dosage form, dissolution controlled-release dosage form, diffusion controlled-release dosage form, controlled-release matrix core, controlled-release matrix core coated with at least one release-slowing coat, enteric coated dosage form, one sustained dosage, dosage form surrounded by at least one delayed-release coat, capsules, minitablets, caplets, uncoated micro particles, micro particles coated with release-slowing coat, micro particles coated with delayed-release coat or any combination thereof. Within the context of this application, the dosage forms described herein mean a dosage form as defined above comprising an effective amount of acetaminophen and an opioid for treating a patient in need of.

The term “effective amount” as used herein means a dosage which is sufficient in order for the treatment of the patient to be effective compared with no treatment.

The term “enhanced absorption dosage forms” or dosage forms which exhibit an “enhanced absorption” of the drug as used herein is defined to mean dosage forms that when exposed to like conditions, will show higher release and/or higher absorption of the drug as compared to other dosage forms with the same or higher amount of drug.

The term “extended release material” as present in the inner solid particulate phase and the outer solid continuous phase refers to one or more hydrophilic polymers and/or one or more hydrophobic polymers and/or one or more other type hydrophobic materials, such as, for example, one or more waxes, fatty alcohols and/or fatty acid esters. The “extended release material” present in the inner solid particulate phase may be the same as or different from the “extended release material” present in the outer solid continuous phase.

The term “extended-release dosage forms” or dosage forms which exhibit an “extended release” of drug as used herein is defined to mean dosage forms administered once daily that release drug slowly, so that plasma concentrations of the drug are maintained at a therapeutic level for an extended period of time such that the sustained-release dosage form provides therapeutic benefit over a 24-hour period.

The term “hydrophilic polymers” as used in this specification include, but are not limited to hydroxypropylmethylcellulose, hydroxypropylcellulose, sodium, carboxymethyl-cellulose, carboxymethylcellulose calcium, ammonium alginate, sodium alginate, potassium alginate, calcium alginate, propylene glycol alginate, alginic acid, polyvinyl alcohol, povidone, carbomer, potassium pectate, potassium pectinate, etc.

The term “hydrophobic polymers” as used in this specification include, but are not limited, to ethyl cellulose, hydroxyethylcellulose, ammonio methacrylate copolymer (Eudragit RL™ or Eudragit RS™), methacrylic acid copolymers (Eudragit L™ or Eudragit S™), methacrylic acid-acrylic acid ethyl ester copolymer (Eudragit L 100-5™), methacrylic acid esters neutral copolymer (Eudragit NE 30D™), dimethylaminoethylmethacrylate-methacrylic acid esters copolymer (Eudragit E 100™), vinyl methyl ether/malefic anhydride copolymers, their salts and esters (Gantrez™) etc.

The term “immediate release coat”, as used herein, is defined to mean a coat, which has substantially or appreciably no influence on the rate of release of acetaminophen or an opioid from the dosage form in-vitro or in-vivo. The excipients comprising the immediate release coat have no substantial slow release, swelling, erosion, dissolution, or erosion and swelling properties, which means that the composition of the coat has no substantial influence on the rate of release of the acetaminophen or an opioid.

The term “instructional material” includes a publication, a recording, a diagram, or any other medium of expression which can be used to communicate the usefulness of the composition of the invention for its designated use. The instructional material of the kit of the invention may, for example, be affixed to a container which contains the composition or be shipped together with a container which contains the composition. Alternatively, the instructional material may be shipped separately from the container with the intention that the instructional material and the composition be used cooperatively by the recipient.

The term “medicament” as used herein means a pharmaceutical dosage form suitable for administration of the pharmaceutically active compound to a patient.

The term “mean maximum plasma concentration” (Cₘₐₓ) as used herein means the arithmetic mean of maximum plasma concentration of acetaminophen or an opioid.

The term “mean plasma concentration” as used herein means the arithmetic mean blood plasma concentration of acetaminophen or opioid.

The term “modified-release dosage forms” or dosage forms which exhibit a “modified-release” of the drug as used herein is defined to mean dosage forms whose drug release characteristics of time course and/or location are designed to accomplish therapeutic or convenience objectives not offered by an immediate-release dosage forms. Modified-release dosage forms or dosage forms are typically designed to provide a quick increase in the plasma concentration of the drug which remains substantially constant within the therapeutic range of the drug for at least a 24-hour period. Alternatively, modified-release dosage forms will desirably be designed to provide a quick increase in the plasma concentration of the drug, which although may not remain constant, declines at rate such that the plasma concentration remains within the therapeutic range for at least a 24-hour period.

The term “multiparticulate” or “microparticle” as used herein is defined to mean a plurality of drug-containing units, such as for example microspheres, spherical particles, microcapsules, particles, micro particles, granules, spheroids, beads, pellets, or spherules.

The term “opioid or opioids or opiates” as used herein means any entity that brings out biological response by acting on opioid receptors. These include but not limited to opioid agonists useful in the present invention include, but are not limited to, alfentanil, axomadol, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, faxeladol, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papavereturn, pentazocine, phenadoxone, phenomorphan, phenazocine, phenoperidine, piminodine, piritramide, propheptazine, promedol, properidine, propoxyphene, sufentanil, tilidine, tapentadol, and, tramadol, mixtures or salts of any of the foregoing.

The term “pain and pain related conditions” as used herein is defined as any pain due to a medical conditions including but not limited to neuropathic pain, osteoarthritis, rheumatoid arthritis, fibromyalgia, and back, musculoskeletal pain, Enclosing spondylitis, juvenile rheumatoid arthritis, migraines, dental pain, abdominal pains, ischemic pain, postoperative pain or because of an anesthetic or surgical contrition.

The term “passage” includes an aperture, orifice, bore, hole, weakened area or a credible element such as a gelatin plug that erodes to form an osmotic passage for the release of the drug from the dosage form.

The term “pharmaceutically acceptable derivative” means various pharmaceutical equivalent isomers, enantiomers, salts, hydrates, polymorphs, esters etc of acetaminophen or an opioid.

The term “prevention of a disease” as used herein is defined as the management and care of an individual at risk of developing the disease prior to the clinical onset of the disease. The purpose of prevention is to combat the development of the disease, condition or disorder, and includes the administration of the active compounds to prevent or delay the onset of the symptoms or complications and to prevent or delay the development of related diseases, conditions or disorders.

The term “swellable polymer,” as used herein, refers to a polymer that will swell in the presence of a fluid. It is understood that a given polymer may or may not swell when present in a defined drug formulation.

The term “slow-release” here applies to any release formulation that is other than an immediate release wherein the release of the active ingredient is slow in nature. This includes various terms used interchangeably in the pharmaceutical context like extended release, delayed release, sustained release, controlled release, timed release, specific release, prolonged release and targeted release etc.

The term “sustained-release dosage forms” or dosage forms which exhibit a “sustained-release” of the drug as used herein is defined to mean dosage forms administered once daily that provide a release of the drug sufficient to provide a therapeutic dose after administration, and then a gradual release over an extended period of time such that the sustained-release dosage form provides therapeutic benefit over a 24-hour period.

The term “treatment of a disease” as used herein means the management and care of a patient having developed the disease, condition or disorder. The purpose of treatment is to combat the disease, condition or disorder. Treatment includes the administration of the active compounds to eliminate or control the disease, condition or disorder as well as to alleviate the symptoms or complications associated with the disease, condition or disorder.

The term “twice daily oral pharmaceutical composition” as used herein is defined as any formulation administered two times a day to a patient in need of.

The term “therapeutically effective amount” means an amount that elicits a biological response in a mammal including the suboptimal amount.

Other hydrophobic materials which may be employed in the inner solid particulate phase and/or outer solid continuous phase include, but are not limited, to waxes such as beeswax, carnauba wax, microcrystalline wax, and ozokerite; fatty alcohols such as cetostearyl alcohol, stearyl alcohol; cetyl alcohol myristyl alcohol etc; and fatty acid esters such as glyceryl monostearate, glycerol monooleate, acetylated monoglycerides, tristearin, tripalmitin, cetyl esters wax, glyceryl palmitostearate, glyceryl behenate, hydrogenated castor oil, etc.

Suitable polymers for use in the present dosage forms may be linear, branched, dendrimeric, or star polymers, and include synthetic hydrophilic polymers as well as semi-synthetic and naturally occurring hydrophilic polymers. The polymers may be homopolymers or copolymers, if copolymers, either random copolymers, block copolymers or graft copolymers. Synthetic hydrophilic polymers useful herein include, but are not limited to: polyalkylene oxides, particularly poly(ethylene oxide), polyethylene glycol and poly(ethylene oxide)-poly(propylene oxide) copolymers; cellulosic polymers; acrylic acid and methacrylic acid polymers, copolymers and esters thereof, preferably formed from acrylic acid, methacrylic acid, methyl acrylate, ethyl acrylate, methyl metbacrylate, ethyl methacrylate, and copolymers thereof, with each other or with additional acrylate species such as aminoethyl acrylate; maleic anhydride copolymers; polymaleic acid; poly(acrylamides) such as polyacrylamide per se, poly(methacrylamide), poly(dimethylacrylamide), and poly(N-isopropyl-acrylamide); poly(olefinic alcohol)s such as poly(vinyl alcohol), poly(N-vinyl lactams) such as poly(vinyl pyrrolidone), poly(N-vinyl caprolactam), and copolymers thereof polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxyethylated propylene glycol, and mono- and di-polyoxyethylated trimethylene glycol; polyoxyethylated sorbitol and polyoxyethylated glucose; polyoxazolines, including poly(methyloxazoline) and poly(ethyloxazoline); polyvinylamines; polyvinylacetates, including polyvinylacetate per se as well as ethylene-vinyl acetate copolymers, polyvinyl acetate phthalate, and the like, polyimines, such as polyethyleneimine; starch and starch-based polymers; polyurethane hydrogels; chitosan; polysaccharide gums; zein; and shellac, ammoniated shellac, shellac-acetyl alcohol, and shellac n-butyl stearate

The term “tapentadol or morphine or hydromorphone or hydrocodone or axomadol or tramadol or faxeladol or oxycodone” as used herein is defined to mean at least one form of tapentadol or morphine or hydromorphone or hydrocodone or axomadol or tramadol or faxeladol or oxycodone, the individually optically active enantiomers of tapentadol or morphine or hydromorphone or hydrocodone or axomadol or tramadol or faxeladol or oxycodone, such as for example, (+) or (−) forms of tapentadol or morphine or hydromorphone or hydrocodone or axomadol or tramadol or faxeladol or oxycodone, racemic mixtures thereof, active metabolites, pharmaceutically acceptable salts thereof, such as for example, acid addition or base addition salts of tapentadol or morphine or hydromorphone or hydrocodone or axomadol or tramadol or faxeladol or oxycodone. Acids commonly employed to form acid addition salts are inorganic acids, such as for example, hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of such pharmaceutically acceptable salts are the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutylate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutylate, citrate, lactate, g-hydroxybutylate, glycolate, tartrate, methanesulfonate, propanesulfonate, naphthalene-1-sulfonate, napththalene-2-sulfonate, mandelate and the like. Base addition salts include those derived from inorganic bases, such as for example, ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like.

The present invention discloses a pharmaceutical dosage form comprising a therapeutically effective amount of at least one opioid, at least one form of acetaminophen and at least one pharmaceutically acceptable excipient wherein the said dosage form is retained in the stomach for at least four hours and is suitable for once daily or twice daily administration. Exemplary Opioids include morphine, hydrocodone, hydromorphone, oxycodone, tramadol, tapentadol, axomadol and faxeladol.

The present invention further provides a pharmaceutical dosage form comprising: a) a core comprising at least two release layers wherein a first release layer comprising at least one opioid with or without acetaminophen, dispersed in a slow-release matrix comprising cross-linked high amylose starch; and a second release layer comprising acetaminophen, with or without an opioid, dispersed in a second-release matrix; and b) a coat comprising the said core. Exemplary Opioids include morphine, hydrocodone, hydromorphone, oxycodone, tramadol, tapentadol, axomadol and faxeladol.

The present invention further provides a pharmaceutical a dosage form comprising a) a compressed core comprising at least two release layers wherein a first release layer comprising at least one opioid, with or without acetaminophen, dispersed in a slow-release matrix; and a second release layer comprising acetaminophen, with or without an opioid, dispersed in a second release matrix; b) at least one permeable membrane covering comprising the said core; c) an encapsulating coat and the said dosage form is suitable for once daily or twice daily administration.

The core includes at least two release layers. The release layers can be a matrix layers and an opioid is slowly released from the matrix. In a specific embodiment, one matrix of the core is a cross-linked high amylose starch prepared according to the standard procedures and described in detail below. The core is formed by mixing the constituents layers and compressing them into a compressed core. The weight of the core could be from about 10% to about 80% of the tablet weight. In the preferred embodiments, the core is from 26% to 33% depending on the amount of opioid and or acetaminophen used to make the tablets. The core comprises opioid with or without acetaminophen dispersed in at least one release layers. The core can also comprise a second release layer comprising acetaminophen, with our without opioid, dispersed in at least one release layer. The opioid in first release layer and second release can be the same or different. The opioid:acetaminophen ratio ranging from about 10:90 to about 90:10 of the total composition.

In yet specific embodiment, about 25% of total opioid amount in the total dosage form is present in at least one release layer.

In yet specific embodiment, about 50% % of total opioid amount in the total dosage form is present in at least one release layer.

In yet specific embodiment, about 75% of total opioid amount in the total dosage form is present in at least one release layer.

In yet specific embodiment, about 100% of total opioid amount in the total dosage form is present in at least one release layer.

In yet specific embodiment, about 25% of total amount of acetaminophen in the total dosage form is present in at least one release layer.

In yet specific embodiment, bout 50% of total amount of acetaminophen in the total dosage form is present in at least one release layer.

In yet specific embodiment, about 75% of total amount of acetaminophen in the total dosage form is present in at least one release layer.

In yet specific embodiment, about 100% of total amount of acetaminophen in the total dosage form is present in at least one release layer.

In specific embodiment, opioid amount in a first release layer is on or around 50% of the total opioid present in the tablet and acetaminophen is on or about 50% of the total acetaminophen present in the total composition.

In yet specific embodiment, opioid amount in a first release layer is on or around 75% of the total opioid present in the tablet and acetaminophen is on or about 25% of the total acetaminophen present in the total composition.

In yet specific embodiment, opioid amount in a first release layer is on or around 25% of the total opioid present in the tablet and acetaminophen is on or about 75% of the total acetaminophen present in the total composition.

In yet specific embodiment, opioid amount in a first release layer is on or around 100% of the total opioid present in the tablet and acetaminophen is on or about 100% of the total acetaminophen present in the total composition.

In specific embodiment, opioid amount in a second release layer is on or around 50% of the total opioid present in the tablet and acetaminophen is on or about 50% of the total acetaminophen present in the total composition.

In yet specific embodiment, opioid amount in a second release layer is on or around 75% of the total opioid present in the tablet and acetaminophen is on or about 25% of the total acetaminophen present in the total composition.

In yet specific embodiment, opioid amount in a second release layer is on or around 25% of the total opioid present in the tablet and acetaminophen is on or about 75% of the total acetaminophen present in the total composition.

In yet specific embodiment, opioid amount in a second release layer is on or around 100% of the total opioid present in the tablet and acetaminophen is on or about 100% of the total acetaminophen present in the total composition.

For example; in one embodiment, 5 mg in a 20 mg of oxycodone dosage formulation is present in at least one release layer.

For example; in one embodiment, 10 mg in a 20 mg of oxycodone dosage formulation is present in at least one release layer.

For example; in one embodiment, 15 mg in a 20 mg of oxycodone dosage formulation is present in at least one release layer.

For example; in one embodiment, all the 20 mg of oxycodone dosage formulation is present in at least one release layer.

For example; in one embodiment, from about 25 to about 100 mg in a 600 mg of acetaminophen present in a dosage formulation is in at least one release layer.

For example; in one embodiment, from about 100 to about 200 mg in a 600 mg of acetaminophen present in a dosage formulation is in at least one release layer.

For example; in one embodiment, from about 200 to about 300 mg in a 600 mg of acetaminophen present in a dosage formulation is in at least one release layer.

For example; in one embodiment, from about 300 to about 400 mg in a 600 mg of acetaminophen present in a dosage formulation is in at least one release layer.

For example; in one embodiment, from about 400 to about 500 mg in a 600 mg of acetaminophen present in a dosage formulation is in at least one release layer.

For example; in one embodiment, from about 500 to about 600 mg in a 600 mg of acetaminophen present in a dosage formulation is in at least one release layer.

In another embodiment of this invention opioid is present at levels ranging from about 1 to about 90 wt. % of the total weight of the core, preferably from about 10 to about 70 wt. % of the total composition of the first release layer, more preferably from about 20 to about 60 wt. % of the total composition of the first release layer, and probably most often between about 30 to about 50 wt. % of the total composition of the first release layer. Opioid is present from about 10% to about 90% of second release layer.

The core also includes at least one matrix and an opioid is slowly released from the matrix. In a specific embodiment, one matrix of the core is a cross-linked high amylose starch prepared according to the standard procedures and described in detail below. The core is formed by mixing the constituents layers and compressing them into a compressed core. The weight of the core could be from about 10% to about 80% of the tablet weight. In the preferred embodiments, the core is from 26% to 33% depending on the amount of an opioid used to make the tablets. An opioid could be from about 10% to about 90% of the total composition. In specific embodiment, Opioid amount in a first release layer is on or around 50% of the total Opioid present in the tablet. For example 50 mg in a 100 mg opioid (tapentadol) dosage or about 50% of a 500 mg acetaminophen dosage formulation.

In specific embodiment, Opioid amount in a first release layer is on or around 50% of the total Opioid present in the tablet. For example 50 mg in a 100 mg opioid (tapentadol) dosage or about 50% of a 500 mg acetaminophen dosage formulation.

Opioid is present at levels ranging from about 1 to about 90 wt. % of the total weight of the core, preferably from about 10 to about 70 wt. % of the total composition of the first release layer, more preferably from about 20 to about 60 wt. % of the total composition of the first release layer, and probably most often between about 30 to about 50 wt. % of the total composition of the first release layer.

In one embodiment at least one matrix of the core is cross-linked high amylose starch and it makes up between about 10% and about 90% by weight of the first release layer. In one particular embodiment, the first release layer totals about 140 mg, of which about 75 mg is cross linked amylose and opioid is about 75 mg thus the matrix makes up about 49 weight percent of the first release layer. However the ratio of the matrix of the first release layer to the active ingredient of the first release layer (w/w) is between about 0.1 and about 10, or between about 0.5 and about 5, or between about 1 and about 4, or between about 1 and about 3 and about 1.5 and about 2.5.

In another embodiment, first release layer is without cross linked amylose and the matrix release opioid slowly.

The first release layer as envisaged in the present invention may optionally include a pharmaceutically acceptable carrier or vehicle flavoring agents; coloring agents; binders; preservatives; lubricants, starch, fillers, glidants, surfactants and the like known to those skilled in the art and are found, for example, in Remington's Pharmaceutical Sciences, 14.sup.th Ed. (1970).

The second release layer of the core includes a physical mixture of polyvinyl acetate and polyvinylpyrrolidone and optionally the active pharmaceutical ingredient(s) of the first release layer. The second layer, prepared by dry compression in a preferred embodiment, can also include a cross-linked high amylose starch. In a particular embodiment described below, polyvinylpyrrolidone making up about 45% by weight of the second layer. The second layer has about 23% of xanthan gum. Opioid is present from about 30% to about 70% by weight of second release layer.

The second release layer of the core can also be without polyvinyl acetate and polyvinylpyrrolidone but comprise pharmaceutical excipients.

The present invention discloses a pharmaceutical dosage form comprising at least one opioid, at least acetaminophen and at least one pharmaceutically acceptable excipient and the dosage form remains in the stomach for at least four hours. According to the invention, the composition preferably contains a therapeutically effective amount of a opioid, therapeutically effective amount of acetaminophen or a pharmaceutically acceptable salt thereof, wherein the opioid is suitably in the range of from 1 to 800 mg depending on the opioid used in the dosage formulation, and acetaminophen is present from about 50 mg to about 1000 mg and the said dosage form is suitable for once daily or twice daily administration. The exact dosage depends on the opioid used in the dosage form. For example; tapentadol could be from about 25 mg to about 200 mg in a dosage form while oxycodone could be from about 2.5 mg to about 100 mg in a dosage form.

The present invention further provides a dosage form comprising: a) a core comprising at least two release layers wherein a first release layer comprising at least one at least one opioid, with or without acetaminophen, is dispersed in a slow-release matrix comprising cross-linked high amylose starch; and a second release layer comprising at least acetaminophen, with or without an opioid, and b) a coat comprising the said core.

The present invention further provides a dosage form comprising: a) a core comprising at least two release layers wherein a first release layer comprising at least one at least one opioid, with or without acetaminophen, is dispersed in a slow-release matrix comprising cross-linked high amylose starch; and a second release layer comprising at least acetaminophen, with or without an opioid, wherein at least one release layer comprises a physical mixture of polyvinyl acetate and polyvinylpyrrolidone, and b) a coat comprising the said core.

The present invention further provides a dosage form comprising: a) a core comprising at least two release layers wherein a first release layer comprising at least one at least one opioid, with or without acetaminophen, is dispersed in a slow-release matrix comprising cross-linked high amylose starch; and a second release layer comprising at least acetaminophen, with or without an opioid, and b) a coat comprising the said core, wherein between 10% and 30% per hour of opioid initially present at 0 hours, is released between 0 and 2 hours when tested in vitro using a USP Type I apparatus in 50 mM phosphate, pH 6.8, and stirring between 50 and 150 rpm

The present invention further provides a dosage form comprising: a) a core comprising at least two release layers wherein a first release layer comprising at least one at least one opioid, with or without acetaminophen, is dispersed in a slow-release matrix comprising cross-linked high amylose starch; and a second release layer comprising at least acetaminophen, with or without an opioid in a second release matrix; b) at least one permeable membrane covering comprising the said core; c) an encapsulating coat comprising the membrane coated core.

The present invention further provides a dosage form comprising: a) a core comprising at least two release layers wherein a first release layer comprising at least one at least one opioid, with or without acetaminophen, is dispersed in a slow-release matrix comprising cross-linked high amylose starch; and a second release layer comprising at least acetaminophen, with or without an opioid in a second release matrix; b) at least one permeable membrane covering comprising the said core; c) an encapsulating coat comprising the membrane coated core, wherein between 10% and 30% per hour of opioid initially present at 0 hours, is released between 0 and 2 hours when tested in vitro using a USP Type I apparatus in 50 mM phosphate, pH 6.8, and stirring between 50 and 150 rpm.

The present invention further provides a dosage form comprising: a core comprising a compressed first release layer comprising cross-linked high amylose starch having an opioid, or a salt thereof, with or without acetaminophen, embedded therein; and a second release layer comprising acetaminophen, with or without an opioid, in a second release matrix. More preferably, the dosage form comprises from about 1 mg to 800 mg of a opioid dispersed in a first release layer comprising a cross-linked high amylose starch; and a second release layer comprising from about 50 mg to about 1000 mg at least acetaminophen in a second release matrix.

The present invention further provides a dosage form comprising: a core comprising a compressed first release layer comprising cross-linked high amylose starch having an opioid, with or without acetaminophen, or a salt thereof, embedded therein; and a second release layer comprising acetaminophen, with or without an opioid, in a second release matrix, wherein the second release matrix is in immediate release form.

The present invention further provides a dosage form comprising: a core comprising a compressed first release layer comprising cross-linked high amylose starch having an opioid, or a salt thereof, with or without acetaminophen, embedded therein; and a second release layer comprising acetaminophen, with or without an opioid, in a second release matrix; and the said second release layer comprising a physical mixture of polyvinyl acetate, polyvinylpyrrolidone and the ratio of the first release/second release layer (w/w) is between about 0.1 and 0.8.

The present invention further provides a dosage form for use for a period of every four hours, or every six hours, every eight hours, every twelve hours, or every eighteen hours, or twenty-four hours, the formulation comprising a compressed core comprising a first release layer comprising at least one opioid, with or without acetaminophen, dispersed in a first slow-release matrix comprising cross-linked high amylose starch, and a second release layer comprising acetaminophen, with or without an opioid, in a second slow-release matrix and a coat comprising the said core.

The invention discloses a dosage form comprising; a) a core comprising at least two release layers wherein a first release layer comprising at least one opioid, with or without acetaminophen, dispersed in a slow-release matrix comprising cross-linked high amylose starch; and a second release layer comprising a physical mixture of polyvinyl acetate, polyvinylpyrrolidone, a binder, acetaminophen, with or without an opioid, wherein the ratio of the first release layer/second release layer (w/w) is between about 0.2 and 0.6; the ratio of polyvinyl acetate/polyvinylpyrrolidone (w/w) is between about 6:4 and 9:1, b) a coat comprising said core.

The invention discloses a dosage form comprising: a) a core comprising at least two release layers wherein a first release layer comprising at least one opioid, with or without acetaminophen, dispersed in a slow-release matrix comprising cross-linked high amylose starch; and a second release layer comprising a physical mixture of polyvinyl acetate, polyvinylpyrrolidone, a binder, acetaminophen, with or without an opioid; wherein the ratio of the first release layer/second release layer (w/w) is between about 0.2 and 0.6; the ratio of the opioid in the first release layer to the opioid in the second release layer is between about 0.7 and about 1, b) a coat comprising said core

In one embodiment, the dosage form comprising a therapeutically effective amount of, at least one opioid, at least one form of acetaminophen and at least one pharmaceutically acceptable excipient may be a bilayer composition that delivers acetaminophen and an opioid over at least twelve hours. The bilayer composition may optionally comprise at least one layer that releases acetaminophen as a rapid-release portion.

It is understood by a person skilled in art that a dosage form of the invention is representative and exemplary. A dosage form may take a variety of shapes and forms, including tablets, caplets or ovoid, and may be coated or uncoated. The preferred form is a tablet.

In at least one embodiment of the invention, includes a dosage form comprising: a) a core comprising at least three release layers wherein an opioid, with or without acetaminophen, is dispersed in at least one layer, and b) a coat comprising the said core.

In at least one embodiment of the invention, includes a dosage form comprising: a) a core comprising at least two release layers wherein a first release layer comprising at least one opioid, with or without acetaminophen, dispersed in a slow-release matrix comprising cross-linked high amylose starch; and a second release layer comprising acetaminophen with or without opioid dispersed in a second release matrix; and a third release layer, optionally comprising either acetaminophen or an opioid, and b) a coat comprising the said core.

In at least one embodiment of the invention, includes a dosage form comprising: a) a core comprising at least two release layers wherein a first release layer comprising at least one opioid, with or without acetaminophen, dispersed in a slow-release matrix comprising cross-linked high amylose starch; and a second release layer comprising acetaminophen, with or without an opioid, dispersed in a second release matrix; b) an opioid antagonist, and c) a coat comprising the said core.

In at least one embodiment of the invention, includes a dosage form comprising: a) a core comprising at least three release layers wherein an opioid, with or without acetaminophen, is dispersed in a slow-release matrix comprising cross-linked high amylose starch; wherein one of the release layer comprises a physical mixture of polyvinyl acetate, polyvinylpyrrolidone, and b) a coat comprising the said core.

In at least one embodiment of the invention, includes a solid dosage formulation comprising an opioid and acetaminophen for release thereof over an extended period of time, the formulation comprises: a) a compressed core comprising at least two release layers wherein a first release layer comprising at least one opioid, with or without acetaminophen, dispersed in a slow-release matrix comprising cross-linked high amylose starch; and a second release layer comprising acetaminophen, with our without an opioid, in a second release matrix; and b) a coat comprising the said core, The exemplary combinations include; tapentadol as the opioid and acetaminophen, tramadol as the opioid and acetaminophen, axomadol as the opioid and acetaminophen, oxycodone as the opioid and acetaminophen, morphine as the opioid and acetaminophen, hydromorphone as the opioid and acetaminophen, hydrocodone as the opioid and acetaminophen, faxeladol as the opioid and acetaminophen.

In at least one embodiment of the invention, includes a solid dosage formulation comprising an opioid and acetaminophen for release thereof over an extended period of time, the formulation comprises: a) a compressed core comprising at least two release layers wherein a first release layer comprising at least one opioid, with or without acetaminophen, dispersed in a slow-release matrix comprising cross-linked high amylose starch; and a second release layer comprising acetaminophen, with our without an opioid, in a second release matrix; and a third release layer, and b) a coat comprising the said core, wherein the said opioid is selected from a group consisting of alfentanil, axomadol, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, faxeladol, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papavereturn, pentazocine, phenadoxone, phenomorphan, phenazocine, phenoperidine, piminodine, piritramide, propheptazine, promedol, properidine, propoxyphene, sufentanil, tilidine, tapentadol, and, tramadol.

In at least one embodiment of the invention, includes a solid dosage formulation comprising an opioid and acetaminophen for release thereof over an extended period of time, the formulation comprises: a) a compressed core comprising at least two release layers wherein a first release layer comprising at least one opioid, with or without acetaminophen, dispersed in a slow-release matrix comprising cross-linked high amylose starch; and a second release layer comprising acetaminophen, with our without an opioid, in a second release matrix; and b) a coat comprising the said core, wherein the said opioid is axomadol,

In at least one embodiment of the invention, includes a solid dosage formulation comprising an opioid and acetaminophen for release thereof over an extended period of time, the formulation comprises: a) a compressed core comprising at least two release layers wherein a first release layer comprising at least one opioid, with or without acetaminophen, dispersed in a slow-release matrix comprising cross-linked high amylose starch; and a second release layer comprising acetaminophen, with our without an opioid, in a second release matrix; and b) a coat comprising the said core, wherein the said opioid is tapentadol.

In at least one embodiment of the invention, includes a solid dosage formulation comprising an opioid and acetaminophen for release thereof over an extended period of time, the formulation comprises: a) a compressed core comprising at least two release layers wherein a first release layer comprising at least one opioid, with or without acetaminophen, dispersed in a slow-release matrix comprising cross-linked high amylose starch; and a second release layer comprising acetaminophen, with our without an opioid, in a second release matrix; and b) a coat comprising the said core, wherein the said opioid is tramadol

In at least one embodiment of the invention, includes a solid dosage formulation comprising an opioid and acetaminophen for release thereof over an extended period of time, the formulation comprises: a) a compressed core comprising at least two release layers wherein a first release layer comprising at least one opioid, with or without acetaminophen, dispersed in a slow-release matrix comprising cross-linked high amylose starch; and a second release layer comprising acetaminophen, with our without an opioid, in a second release matrix; and b) a coat comprising the said core, wherein the said opioid is morphine

In at least one embodiment of the invention, includes a solid dosage formulation comprising an opioid and acetaminophen for release thereof over an extended period of time, the formulation comprises: a) a compressed core comprising at least two release layers wherein a first release layer comprising at least one opioid, with or without acetaminophen, dispersed in a slow-release matrix comprising cross-linked high amylose starch; and a second release layer comprising acetaminophen, with our without an opioid, in a second release matrix; and b) a coat comprising the said core, wherein the said opioid is faxeladol.

In at least one embodiment of the invention, includes a solid dosage formulation comprising an opioid and acetaminophen for release thereof over an extended period of time, the formulation comprises: a) a compressed core comprising at least two release layers wherein a first release layer comprising at least one opioid, with or without acetaminophen, dispersed in a slow-release matrix comprising cross-linked high amylose starch; and a second release layer comprising acetaminophen, with our without an opioid, in a second release matrix; and b) a coat comprising the said core, wherein the said opioid is oxycodone.

In at least one embodiment of the invention, includes a solid dosage formulation comprising an opioid and acetaminophen for release thereof over an extended period of time, the formulation comprises a) a compressed core comprising at least two release layers wherein a first release layer comprising at least one opioid, with or without acetaminophen, dispersed in a slow-release matrix comprising cross-linked high amylose starch; and a second release layer comprising acetaminophen, with our without an opioid, in a second release matrix; b) at least one permeable membrane pouch or a sachet comprising the said core; c) an encapsulating coat and exemplary opioid is axomadol, hydromorphone, hydrocodone, tapentadol, morphine, oxycodone, tramadol and faxeladol

In one embodiment, the dosage form uses an expansible and permeable membrane is used to house the core. The membrane can absorb body fluid, such as gastric juice, and can affect a slow and continuous release of controlled amounts of the acetaminophen or opioid by means of diffusion or optionally by the use of osmosis. Suitable plastic or wax-like polymeric materials are especially hydrophilic materials such as methyl- or ethyl-cellulose, hydroxypropylcellulose, methyl- or ethyl-hydroxyethylcellulose, methyl- or ethyl-hydroxypropylcellulose, carboxymethylcellulose, polyvinyl acetate, polyvinylpyrrolidone, polyacrylonitrile, mixtures of polyvinylpyrrolidone with polyvinyl alcohol, resins based on phthalic acid anhydride/polyhydroxy alcohol, urethanes, polyamides, shellac, etc. The preferred are fully hydrolysed polyvinyl alcohol (more than 97%) is preferred. The membranes can be pre-formed pouch or a sachet.

Such membrane coated cores are provided with a disintegrating coat, upon contact with body fluids, is provided using suitable film coating materials. The covering materials include hydrophilic cellulose derivatives, such as cellulose ethers-methylcellulose, hydroxypropylcellulose or especially hydroxypropylmethylcellulose, mixtures of polyvinylpyrrolidone or of a copolymer of polyvinylpyrrolidone and polyvinyl acetate with hydroxypropylmethylcellulose, mixtures of shellac with hydroxypropylmethylcellulose, polyvinyl acetate or copolymers thereof with polyvinylpyrrolidone, or mixtures of water-soluble cellulose derivatives, such as hydroxypropylmethylcellulose, and water-insoluble ethyl cellulose can be used. It is also possible to substitute the covering with a hard gelatin capsules.

A further embodiment of this invention is to provide an oral dosage form that can be administered orally or rectal or sublingual or buccal.

Granules, spheroids, pellets, multiparticulates, capsules, patches tablets, sachets, controlled release suspensions, or in any other suitable dosage form incorporating such granules, spheroids, pellets or multiparticulates are also a part of the present invention.

Granules, spheroids, pellets, multiparticulates, capsules, patches tablets, sachets, controlled release suspensions, or in any other suitable dosage form incorporating such granules, spheroids, pellets or multiparticulates are also a part of the present invention.

The one or more of active ingredient in the composition according to the present invention may suitably be incorporated in a matrix. This may be any matrix, known to a person skilled the art, that affords slow release tapentadol over at least a twelve hour period and preferably that affords in-vitro dissolution rates and in vivo absorption rates of tapentadol within the therapeutically effective ranges. The formulation according to the present invention may preferably use a slow release matrix. Alternatively, normal release matrices having a coating which provides for slow release of the tapentadol may be used. This may be any matrix that affords tapentadol released over at least a twelve hour period and preferably that affords in-vitro dissolution rates and in vivo absorption rates of tapentadol within the ranges specified above. Preferably the matrix is a controlled release matrix. Alternatively, normal release matrices having a coating which provides for controlled release of the active ingredient may be used.

The slow release matrix employed in the composition of this invention may also contain other pharmaceutically acceptable ingredients which are conventional in the pharmaceutical art such as diluents, lubricants, binders, granulating aids, colorants, flavourants, surfactants, pH adjusters, anti-adherents and glidants, e.g. dibutyl sebacate, ammonium hydroxide, oleic acid and colloidal silica. Any known diluent e.g. microcrystalline cellulose, lactose and dicalcium phosphate may be used to prepare this combination. Suitable lubricants are e.g. magnesium stearate and sodium stearyl fumarate. Suitable binding agents are e.g. hydroxypropyl methyl cellulose, polyvidone and methyl cellulose. Suitable disintegrating agents are starch, sodium starch glycolate, and Crospovidone and Croscarmellose sodium.

Slow release matrix of present invention includes materials such as Polyalkylene glycols, Long Chain Hydrocarbons and Hydrophilic or hydrophobic polymers, such as gums, cellulose ethers, acrylic resins and protein derived materials. Of these polymers, the cellulose ethers, especially alkyl celluloses are preferred. The preparation may conveniently contain between 1% and 80% (by weight) of one or more hydrophilic or hydrophobic polymers. Still further Digestible, long chain (C.8-050), substituted or un-substituted hydrocarbons, such as fatty acids, fatty alcohols, glyceryl esters of fatty acids, mineral and vegetable oils and waxes, Of these long chain hydrocarbon materials, fatty (aliphatic) alcohols are preferred. The preparation may conveniently contain up to 60% (by weight) of at least one digestible, long chain hydrocarbon.

Surface actives that are suitable for this invention are Poloxamer 188®, polysorbate 80 and sodium lauryl sulfate. The suitable flow aids for this invention are talc colloidal anhydrous silica. Similarly, the suitable water soluble polymers that may be used to prepare the matrix are PEG with molecular weights in the range 1000 to 6000. The combination comprising an opioid and acetaminophen according to the invention may conveniently be film coated using any film coating material conventional in the pharmaceutical art but preferably an aqueous film coating is used.

Alternatively, the composition as per this invention may comprise a normal release matrix having a slow release coating. Preferably the combination comprises film coated spheroids containing the active ingredient and a spheronising agent. The spheronising agent may be any suitable pharmaceutically acceptable material which may be spheronised together with the active ingredient to form spheroids. A preferred spheronising agent as per this invention is microcrystalline cellulose. The microcrystalline cellulose used may suitably be, for example, Avicel PH 101 or Avicel PH 102 (Trade Marks, FMC Corporation). The spheroids may optionally contain other pharmaceutically acceptable ingredients conventional in the pharmaceutical art such as binders, bulking agents and colorants. Suitable binders may include water soluble polymers, water soluble hydroxyalkyl celluloses such as hydroxypropylcellulose or water insoluble polymers (which may also contribute controlled release properties) such as acrylic polymers or copolymers for example ethyl cellulose. Suitable bulking agents include lactose.

The spheroids are coated with a material which permits release of the active ingredient at a slow rate in an aqueous medium. Suitable slow release coating materials that may be used in this invention include water insoluble waxes and polymers such as polymethylacrylates (for example Eudragit polymers, Trade Mark) or water insoluble celluloses, particularly ethyl cellulose. Optionally, water soluble polymers such as polyvinylpyrrolidone or water soluble celluloses such as hydroxypropylmethylcellulose or hydroxypropylcellulose may be included. Optionally other water soluble agents such as polysorbate 80 may be added.

Further in an alternative embodiment, a flux-enhancing agent can also be included in the membrane or slow release coating can include one of the above-described polymers. The flux enhancing agent can increase the volume of fluid imbibed into the core to enable the dosage form to dispense substantially all of opioid through the passage and/or the porous membrane. The flux-enhancing agent can be a water-soluble material or an enteric material. Examples of the preferred materials that are useful as flux enhancers include but not limited to sodium chloride, potassium chloride, sucrose, sorbitol, mannitol, polyethylene glycols (PEG), propylene glycol, hydroxypropyl cellulose, hydroxypropyl methylcellulose, hydroxypropyl methylcellulose phthalate, cellulose acetate phthalate, polyvinyl alcohols, methacrylic acid copolymers, poloxamers (such as LUTROL F68, LUTROL F127, LUTROL F108 which are commercially available from BASF) and mixtures thereof. A preferred flux-enhancer used in this invention is PEG 400.

A commonly known excipient such as a plasticizer may also be used for preparing the membrane or slow release coating Some commonly known plasticizers include but not limited to adipate, azelate, enzoate, citrate, stearate, isoebucate, sebacate, triethyl citrate, tri-n-butyl citrate, acetyl tri-n-butyl citrate, citric acid esters, and all those described in the Encyclopedia of Polymer Science and Technology, Vol. 10 (1969), published by John Wiley & Sons. The preferred plasticizers are triacetin, acetylated monoglyceride, grape seed oil, olive oil, sesame oil, acetyltributylcitrate, acetyltriethylcitrate, glycerin sorbitol, diethyloxalate, diethylmalate, diethylfumarate, dibutylsuccinate, diethylmalonate, dioctylphthalate, dibutyl sebacate, triethyl citrate, tributyl citrate, glycerol tributyrate and the like. Though the exact amount used depends on the type of plasticizer used, typically amounts from about 0 to about 25% are used, and preferably about 2% to about 15% of the plasticizer can be used based upon the total weight of the membrane or sustained release coating.

Generally, the membrane or slow release coating around the core will comprise from about 1% to about 20% and preferably about 2% to about 10% based upon the total weight of the core and coating.

The membrane or sustained release coating surrounding the core can further optionally comprise a passage that will allow for controlled release of the drug from the core in a preferred embodiment. As used herein the term passage includes an aperture, orifice, bore, hole, weakened area or a credible element such as a gelatin plug that erodes to form an osmotic passage for the release of the tapentadol from the dosage form. The passage used in accordance with the subject invention is well known and such passages are described in U.S. Pat. Nos. 3,845,770; 3,916,899; 4,034,758; 4,077,407; 4,783,337 and 5,071,607.

In another set of embodiments, the invention includes a bilayer formulation wherein the first layer defining a fast release portion of the composition and comprising at least one form acetaminophen; and a second layer defining a slow release portion of the composition and comprising at least one form of acetaminophen and at least one form of an opioid and at least one pharmaceutical excipient, wherein the dosage form is suitable for twice daily administration. Administration of the said dosage form provides rapid analgesia within an hour of administration and lasts up to twelve hours after administration. The acetaminophen release for the said dosage form corresponds to the following: between 30% and 50% acetaminophen released after 1 hour; between 60 and 90% acetaminophen released after 4 hours; between 60% and 90% acetaminophen released after 8 hours; not less than 90% acetaminophen is released after 12 hours

The following examples are shown for illustrating the invention related to a dosage form comprising at least one form of an opioid and at least one form of acetaminophen with pharmaceutically acceptable carrier. These examples in no way limit the scope of the invention. The person skilled in the art will know how the combination may be modified using other formulations and excipients.

### EXAMPLES

### Example 1

### Tapentadol Core

A tapentadol core containing 50 mg of tapentadol HCl is prepared using the preparations methods known in the art with the following formula in Table 1;

### Example 1

TABLE 1

| Core | mg/tablet |
|---|---|
| Tapentadol Hydrochloride | 50 |
| Lactose Monohydrate | 247 |
| Silicon Dioxide | 1.5 |
| Magnesium Stearate | 1.5 |
| Total | 300 |
| Seal Coat (optional) | |

### Manufacturing Process

A 50 mg Tapentadol core was prepared using standard techniques. Specifically, the tapentadol HCl, lactose, colloidal silicon dioxide and magnesium stearate are delumped by passing them through a 40 mesh screen. The delumped materials, tapentadol HCl, lactose, and colloidal silicon dioxide, are then blended for approximately thirty (30) minutes in a suitable blender. The delumped magnesium stearate is then added to the blender and blended for five (5) minutes. After blending, the mixture is compressed on a rotary press with tooling that has an indentation. Optionally a seal coating was applied using standard techniques known in art with an Opadry material or other suitable water-soluble coating material. Opadry was dissolved in water to prepare an Opadry coating solution that was sprayed in a pan coater under standard conditions.

The tablet dosage core comprising tapentadol 50 mg prepared according to the present invention, For Example 1, should exhibit the following dissolution profile when tested in a USP type 1 apparatus at 100 rpms in 900 ml (pH 7.5 phosphate buffer) and at 37′ C.: between 0 and 100% tapentadol released after 2 hour; not less than 50% tapentadol released after 4 hours; more preferably, between 30 and 100% tapentadol released after 2 hours; more preferably, not less than 70% tapentadol released after 4 hours;

### Example 2

### Slow Release Tapentadol

Another embodiment of a slow release tablet containing 50 mg of tapentadol HCl is prepared by first preparing a core as described in Example 1. The core of Example 1 is then coated with a slow release coating according to formula of Table 2;

### Example 2

TABLE 2

| Core | mg/tablet |
|---|---|
| Tapentadol Hydrochloride | 50 |
| Lactose Monohydrate | 247 |
| Silicon Dioxide | 1.5 |
| Magnesium Stearate | 1.5 |
| Total | 300 |
| Seal Coat (optional) | |
| Membrane Coat | |
| Cellulose Acetate | 25.8 |
| Eudragit S100 | 8.6 |
| Triacetin | 2.5 |
| PEG 400 | 2.5 |
| Sugar | 5.1 |
| Total Weight | 344.5 |

### Manufacturing Process

The slow release tablet of Example 2 is prepared following formula of Table 2 using well established manufacturing methods, to coat the seal coated immediate release tablet core prepared according to Example 1, known in art. Specifically, Eudragit 5100, Cellulose Acetate, Triacetin and PEG 400 are dissolved in acetone and Isopropyl alcohol mixture. The polymer solution was homogenized with sugar and the suspension was sprayed over the seal coated immediate release 50 mg tapentadol hydrochloride tablets prepared according Example 1 at coating conditions of 26-32′ C.; atomization pressure of approximately 3 bars; and spray rate of 15-35 ml/min. The sealed core tablet is coated until a theoretical coating level of approximately 12.5% is obtained.

The slow release dosage tablets or pellets, prepared according to the present invention, For Example 2, should exhibit the following dissolution profile when tested in a USP type 1 apparatus at 100 rpms in 900 ml of pH 7.5 phosphate buffer and at 37′ C.: between 0 and 20% tapentadol released after 2 hour; between 5 and 40% tapentadol released after 4 hours; between 30 and 75% tapentadol released after 8 hours; not less than 50% tapentadol released after 8 hours; not less than 60% tapentadol released after 16 hours; not less than 70% tapentadol released after 20 hours, more preferably, between 0 and 10% tapentadol released after 2 hour; more preferably, between 10 and 35% tapentadol released after 4 hours; more preferably, between 40 and 70% tapentadol released after 8 hours; more preferably, not less than 60% tapentadol released after 8 hours; more preferably, not less than 70% tapentadol released after 16 hours; more preferably, not less than 80% tapentadol released after 20 hours, by weight.

### Example 3

### Tapentadol and Acetaminophen Capsules

TABLE 3

| Tapentadol Core | mg/tablet |
|---|---|
| Tapentadol Hydrochloride | 50 |
| Lactose Monohydrate | 247 |
| Silicon Dioxide | 1.5 |
| Magnesium Stearate | 1.5 |
| Total | 300 |
| Seal Coat (optional) | |
| Slow Release Coat | |
| Cellulose Acetate | 25.8 |
| Eudragit S100 | 8.6 |
| Triacetin | 2.5 |
| PEG 400 | 2.5 |
| Sugar | 5.1 |
| Tapentadol Pellets | 344.5 |

Acetaminophen Pellets

| | |
|---|---|
| Acetaminophen | 300 |
| Eudragit RSPO | 88 |
| ETHOCEL (Ethocel ® PR 100) | 4.5 |
| Stearyl Alcohol | 35 |
| Acetaminophen Pellets | 427.5 |
| Total Tapentadol and Acetaminophen | 772 |
| Capsules | |

Acetaminophen, Eudragit and ETHOCEL are blended together in a blender. To the well blended mix, milled stearyl alcohol is added and the contents were thoroughly mixed together and fed an extruder and later a pelletizer. The pellets are screened and sieved to obtain the required acetaminophen pellets. The final capsules comprising tapentadol, prepared according to Example 2 and acetaminophen pellets, prepared as described above, were prepared by filling the required quantity of tapentadol pellets and acetaminophen pellets and the final formula as per Table 3.

### Example 4

| Example 4 | mg/Tablet |
|---|---|
| Tapentadol Hydrochloride | 30 |
| Acetaminophen | 300 |
| Poloxamer | 10 |
| Povidone | 10 |
| Sodium Starch Glycolate | 26 |
| Avicel PH 101 NF | 38.5 |
| Lactose | 30 |
| Stearic Acid | 3 |
| Cab-O-Sit NF | 1 |
| Total Weight of Coated Capsules | 418.5 |

### Manufacturing Process;

A capsule formulation, containing 50 mg tapentadol and 300 mg acetaminophen was made according to the following procedure using the following materials in the indicated amounts expressed in mg/capsules.

50 mg tapentadol, USP; 300 mg acetaminophen, USP; 10.0 mg citric acid (anhydrous); and 11.0 mg Poloxamer were mixed for one minute in a high sheer mixer granulator. The resulting essentially homogenous formulation blend was passed through a Hammer mill (Fitz Mill) fitted with a screen 0.0027″ openings (no. 1532-0027) with the hammer forward on medium speed. The homogenous formulation blend was then mixed for one minute in the high shear mixer granulator.

Separately, 10.0 mg povidone was passed through a 30 mesh (US Std) screen and then added to the homogenous formulation blend, where the resulting mixture was mixed for thirty seconds. Subsequently and separately, 26.0 mg sodium starch glycolate (Primojel) was passed through the hammer mill (Fitz Mill) fitted with a screen 0.0027″ openings (screen no. 1532-0027) and added to the mixture. Further, 30.0 mg lactose monohydrate, NF, together with 38.5 mg Avicel PH 101, NF, were passed through a no. 30 mesh screen and then added to the mixture. The mixture was then mixed for two minutes in a high shear mixer granulator. 3.0 mg stearic acid, NF, and 1.0 mg Cab-O—Sil, NF, were deagglomerated by being passed through a 30 mesh screen. The mixture was then mixed for thirty seconds in a high sheer mixer granulator to form 164 mg of the final formulation product. The final formulation product was then encapsulated using a capsule machine in hard gelatin capsules, size no. 4.

### Example 5

TABLE 5

| | mg/Tablet |
|---|---|
| Tapentadol Hydrochloride | 30 |
| Acetaminophen | 300 |
| Cellulose | 45 |
| Pre-gelatinized Starch | 14 |
| Sodium Starch Glycolate | 15 |
| Starch | 28.5 |
| Water | 140 |
| Magnesium Stearate | 1.5 |
| Coat | |
| Opadry Light Yellow | 2 |
| Carnauba wax | 5 |
| Total Weight of Coated Tablets | 441 |

### Manufacturing Process

The ingredients of Table 5 were mixed and sieved and the samples were taken to assess homogeneity. The powder was compressed with suitable punches for the different tablets sizes (hardness was adjusted in order to obtain friability of <1% after 400 rev.). Tablets were spray-coated with a target weight increase of 4.0%, with Opadry® II (Colorcon, West Point, Pa., USA), if needed different colors for the different doses.

### Example 6

A reference sample of 300 mg Acetaminophen immediate release tablets was prepared according to standard procedures as per the formula in Table 6.

TABLE 6

| | mg/Tablet |
|---|---|
| Acetaminophen | 300 |
| PVP | 45 |
| Lactose, Hydrogen Dried | 125 |
| Magnesium Stearate | 1.5 |
| Aquacoat ECD (as 30% W/W | 16.5 |
| suspension) | |
| Methocel | 16.5 |
| Triethyl Citrate | 7.5 |
| Water As required | |
| Total Weight of Coated Tablets | 504.5 |

### Examples 7-19

The Examples 7-17 were formulated using cross-linked amylose and the general manufacturing process in exemplified below.

### Manufacturing Process: Cross-Linked Amylose

The cross-linking of amylose is well known in the literature and the desired cross-linking of amylase can be carried out using the methods described in BIOCHIMIE 1978, 60, 535-537. The cross-linking of amylose is well-known in the literature. For example, the desired cross-linking can be controlled in the manner described by Mateescu et al. in Analytical Letters, 1985, 18, 79-91, by reacting amylose with epichlorohydrin in an alkaline medium. For Example; Cross-linked amylose is produced by reaction of amylose with a cross-linking agent such as epichlorohydrin, in an alkaline medium. Similarly, in the same manner, amylose can also be cross-linked with 2,3-dibromopropanol.

U.S. Pat. No. 5,456,921 discloses cross-linked amylose having a cross-linking degree ranging from 1 to 10 and is known to be particularly useful as a controlled release excipient for the preparation of tablets by direct compression It is also known (W094/02121) that α-amylase can be incorporated into tablets made of cross-linked amylose in order to increase the dissolution rate of low soluble drugs.

Cross-linked amylose having a cross-linking degree of 6 to 30 is further known (WO94/21236) to be useful as a binder and/or disintegrant excipient for the preparation of tablets by direct compression. The binding properties of this product are reported to be definitively superior to starch. The quality of the binding and the controlled release properties of cross-linked amylose are closely related to the cross-linking degree and to the relative amount of amylose present in the starch used for the manufacture.

In all these patent and laid-open applications described above, a laboratory scale process of manufacture of cross-linked amylose is disclosed, which consists of reacting in a planetary mixer a product distributed by Sigma Chemicals, and consists of a corn starch containing more than 70% of amylose w/w, with epichlorohydrin in an alkaline medium. The obtained product is washed on a Buchner funnel with a solution of acetone and dried with pure acetone. About 40 Kg of acetone are needed to manufacture 1 Kg of cross-linked high amylose starch. It is well known in the art that the use of alcohols and/or acetone for the treatment of starch is reported to complex the amylose fraction.

Different degrees of cross-linking can be obtained by varying the ratio of epichlorohydrin to amylose in the reaction vessel. Tablets prepared by direct compression of a dry mixture of cross-linked amylose and a drug swell in solution and show a sustained release of the drug. Depending on the degree of cross-linking of the matrix, different degrees of swelling are obtained. Increasing the degree of cross-linking of amylose first generates an increase of drug-release time, followed by a decrease of drug-release time. The peak drug-release time is observed at a cross-linking degree value of 7.5. A further increase in the degree of cross-linking leads to an accelerated drug release from the cross-linked amylose tablets as a consequence of the erosion process. For cross-linking degree equal or greater than 7.5, increasing the degree of cross-linking of amylose generates a decrease of drug-release time. With degrees of cross-linking above 11, the swollen polymeric matrix presents in vitro disintegration over a period of approximately 90 minutes. The present inventors surprisingly found that a polymer of amylose cross-linked with a cross-linking agent selected from 2,3-dibromopropanol and epichlorohydrin, wherein from about 0.1 to about 10 g of cross-linking agent was used to cross-link 100 g of amylose, is suitable for preparing slow release formulations comprising acetaminophen, at least one opioid, and at least one pharmaceutically acceptable excipient.

Illustrative Manufacturing Process: The process includes gelatinization, cross linking the gelatinized high amylase starch, removal of by-products and thermal treatment to obtain cross-linked amylase desired properties.

A slurry containing 1.2 KG of high amylase starch was prepared by mixing 2.65 KG of water and slurry was thoroughly mixed. To the slurry, 1.97 KG of sodium hydroxide solution at 11.9% w/w was introduced under. The gelatinization was carried out 50′ C. for 20 minutes in a 200 L GOAVEC® crystallization tank. Under intensive stirring, 50 G of epichlorohydrin was introduced into the 1.2 KG of the gelatinized high amylose starch recovered in the previous step. The reaction was carried out at 50′ C for an hour. After reaction, the reaction medium was diluted with 5 KG of water at 60′ C and the mixture was neutralized with an acetic acid solution (37.5% w/w) to obtain a pH below 8. The neutralized product was diluted with 5 KG of water at 50′ C. And cooled down and retained at 4′ C. The product recovered from the previous step was diluted under agitation with 10 KG of water at 50′ C. A diafiltration was realized with an ALFA-LAVAL© apparatus model UFS-6 equipped with 6 hollow fiber polysulfone membrane of 60 mils opening and surface of 25 square feet with pore sizes of 50000 Da. An average of 50 KG Kg of water at 50′ C was used to remove all the by-products such as sodium acetate. Then, the resulting product was concentrated up to 3.8% w/w by ultra filtration and the p. The recovered product was cooled down to 4′ C and was maintained at that temperature until the next step. As briefly discussed hereinabove, the properties of the prepared cross-linked high amylose starch that are required to make it useful as an excipient for drug controlled release are surprisingly dependent to the thermal treatment applied to the slurry just before spray drying. In order to demonstrate this dependency, cross-linked high amylose starch prepared as disclosed hereinabove was treated at different temperatures (100′ C to 50′ C) and the preferred temperature is 90′ C. The cross linked amylase slurry prepared as above was heated to 90′ C at constant stirring for about 5 minutes. Then, the reactant was cooled down to 50′ C under stirring and spray dried at 3.8% of solids in a Niro spray dryer model P6.3 of water evaporating capacity of 50 KG/HOUR, equipped with a atomizer disc and having an inlet temperature of 300′ C and an outlet temperature of 120′ C. The dry cross-linked amylose powder is a controlled release excipient suitable for preparing pharmaceutical dosage form comprising acetaminophen, an opioid such as morphine or tapentadol or oxycodone and at least one pharmaceutical excipient, wherein the dosage form is suitable for once daily or twice daily administration.

### Manufacturing Process: Core

### First Release Layer:

The a pharmaceutical dosage form comprising: a) a core comprising at least two release layers wherein a first release layer comprising at least one opioid with or without acetaminophen dispersed in a slow-release matrix comprising cross-linked high amylose starch; and a second release layer comprising acetaminophen with or without opioid dispersed in a slow-release matrix; and b) a coat comprising the said core. In a specific embodiment, the matrix of the core is a cross-linked high amylose starch prepared according to the process described above.

The first-release matrix is formed by mixing the ingredients and then compressing the mixture to form the first-release matrix layer. The weight of the first-release matrix can be from about 10% to about 80% of the dosage form. In specific embodiments, the matrix makes up between about 10% and about 90% by weight of the first-release matrix layer i.e., the ratio of the matrix of the first-release layer to the active ingredient of the first-release matrix layer (w/w) is between about 0.1 and about 10, or between about 0.2 and about 9, or between about 0.2 and about 8, or between about 0.3 and about 7, or between about 0.4 and about 6, or between about 0.5 and about 5, or between about 0.6 and about 4, or between about 0.7 and about 4 or between about 1 and about 4, or between about 1 and about 3 and about 1.5 and about 2.5.

Optionally the carriers or vehicles are known to those skilled in the art and are found, for example, in Remington's Pharmaceutical Sciences, 14^{th} Ed. (1970) can be optionally included in the core. These include other suitable binders, glidants, lubricants, dyes, sweetening, microcrystalline cellulose, starch, cross-linked starch, cross-linked poly(vinyl pyrrolidone), and sodium carboxymethyl cellulose; flavoring agents; coloring agents; binders; preservatives; surfactants or flavoring agents can also be included.

Example; Cross linked amylose prepared according to the process described above was mixed with colloidal silicon dioxide and passed through a #30 mesh screener. Similarly, cross linked amylose prepared according to the process described above was mixed with acetaminophen, in a blender after passing through a #30 mesh screener. The Magnesium Stearate and Hydrogenated Vegetable Oil Type I are sieved through a #30 mesh screen separately and add to the blender. The cross linked amylose and colloidal silicon dioxide blend was blended with cross linked amylose-acetaminophen blend and hydrogenated vegetable oil through a #30 mesh screen and add blend with other ingredients. This constitutes a first release layer.

### Second-Release Layer:

This second-release matrix layer includes a physical mixture of polyvinyl acetate and polyvinylpyrrolidone and the active pharmaceutical ingredient(s) of the second-release matrix layer with tapentadol. The second-release matrix can also include a cross-linked high amylose starch prepared as described above and other optional components. The weight of the second-release matrix layer can be any percentage of the weight of the total composition between about 10% and about 90% such that it is between about 20% to about 90%, (w/w) of a tablet of the invention, or about 25% to about 90%, or about 30% to about 85%, or about 35% to about 85%, or about 40% to about 85%, or about 45% to about 85%, or about 45% to about 90%, or about 50% to about 90% or about 50% to about 85%, or about 55% to about 90%, or about 55% to about 85%, or about 55% to about 80%, or about 60% to about 90%, or about 60% to about 85%, or about 60% to about 80%, or about 60% to about 75%, or about 65% to about 90%, or about 65% to about 85%, or about 65% to about 80%, or about 65% to about 75%, or about 65% or about 70% or about 75%.

The weight percentage of the polyvinyl acetate/polyvinylpyrrolidone mixture in the second-release matrix layer can have a wide range of values. In particular the polyvinyl acetate/polyvinylpyrrolidone mixture can be from about 10 to about 90 wt. % of the second-release matrix layer, preferably from about 20 to about 80 wt. %, or about 30 to about 60 wt. %. In a particular embodiment of this invention, Kollidon© SR makes up from about 45% by weight of a second-release matrix that is about 31% by weight acetaminophen (or opioid) and about 23% xanthan gum.

The weight ratio of polyvinyl acetate to polyvinylpyrrolidone in the polyvinyl acetate/polyvinylpyrrolidone mixture can be a wide range of values. Preferably, such ratio is between from about 6:4 and 9:1; more likely between from about 7:3 and 6:1, even more preferably about 8:2. The molecular weight of the polyvinyl acetate component in the polyvinyl acetate/polyvinylpyrrolidone mixture can have a wide range of values. For Example, the average molecular weight of the polyvinyl acetate is about 100 to about 10,000,000; or about 1,000 to about 1,000,000; or about 10,000 to about 1,000,000; or about 100,000 to about 1,000,000; or about 450,000. Similarly, the average molecular weight of the polyvinylpyrrolidone can be from about 100 to about 10,000,000; or from about 1,000 to about 1,000,000; or from about 5,000 to about 500,000; or from about 10,000 to about 100,000; or about 50,000.

The polyvinyl acetate and polyvinylpyrrolidone mixture can be prepared by a variety of processes as described the art well known to person skilled in pharmaceutical art. For example, it can be prepared by simply mixing powders of polyvinylpyrrolidone and polyvinyl acetate and other ingredients. In a preferred embodiment of this invention, such mixture is spray dried powder of a colloidal dispersion of polyvinyl acetate and polyvinylpyrrolidone solution. This admixture can also be added optionally stabilizers glidants etc. Optionally the carriers or vehicles are known to those skilled in the art and are found, for example binders, glidants, lubricants, dyes, sweetening, microcrystalline cellulose, starch, cross-linked starch, cross-linked poly(vinyl pyrrolidone), and sodium carboxymethyl cellulose; flavoring agents; coloring agents; binders; preservatives; surfactants or flavoring agents can also be included. Suitable binding agents for the present invention include, but are not limited to, plant extracts, gums, synthetic or natural polysaccharides, polypeptides, alginates, synthetic polymers, or a mixture thereof. There can be easily found in Remington's Pharmaceutical Sciences, 14.sup.th Ed. (1970). These include other suitable plant extracts to be used as gelling agents include, but are not limited to, agar, ispaghula, psyllium, cydonia, ceratonia or a mixture thereof. Suitable synthetic polymers to be used as gelling agents include, but are not limited to, carboxyvinyl polymer, polyvinyl alcohol, polyvinyl pyrrolidone, polyethelene oxide, polyethylene glycols, copolymers of ethylene oxide and propylene oxide and their copolymers or a mixture thereof. In a preferred embodiment of this invention, the gelling agent is a gum such as xanthan gum, guar gum, acacia gum, ghatti gum, karaya gum, tragacanth gum or a mixture thereof, PEO 7,000,000 and HPMC K100 M. In a most preferred embodiment of this invention, xanthan gum is use.

Place a portion of the KollidonR™ SR in a blender and mix with colloidal silicon dioxide, an opioid such as tapentadol, after passing through Kason Separator with a #30 mesh screener, Xanthan gum and hydrogenated vegetable oil Type 1 that is sieved through a #30 mesh screen were added to a blender and all ingredients were blended together. The magnesium stearate sieved through a #30 mesh screen and blended with other ingredients to prepare the second release layer.

This constitutes a second release layer.

### Third Release Layer (Optional);

The third release layer is optional and can be easily prepared according to procedures known in the art. The active agent, which is optional, swellable polymers, diluents and other additives may be mixed and further processed by either dry, wet granulation or direct compression. For Example, in one embodiment of this invention, Microcrystalline Cellulose, Crospovidone, Silicon Dioxide, Magnesium Stearate and Polyvinyl Pyyrolidone were mixed in a mixer and compressed into a third release layer. The third release layer can comprise an opioid such as tapentadol as an immediate release layer.

In another embodiment of this invention, a dosage form comprising tapentadol and acetaminophen, a third layer is formed by mixing acetaminophen, Microcrystalline Cellulose, Crospovidone, Silicon Dioxide, Magnesium Stearate and Polyvinyl Pyyrolidone in a mixer and compressed into a third release layer to form an immediate release layer.

In another embodiment of this invention, a dosage form comprising oxycodone and acetaminophen, a third layer is formed by mixing acetaminophen, Microcrystalline Cellulose, Crospovidone, Silicon Dioxide, Magnesium Stearate and Polyvinyl Pyyrolidone in a mixer and compressed into a third release layer to form an immediate release layer.

In another embodiment of this invention, a dosage form comprising morphine and acetaminophen, a third layer is formed by mixing acetaminophen, Microcrystalline Cellulose, Crospovidone, Silicon Dioxide, Magnesium Stearate and Polyvinyl Pyyrolidone in a mixer and compressed into a third release layer to form an immediate release layer.

In another embodiment of this invention, a dosage form comprising axomadol and acetaminophen, a third layer is formed by mixing acetaminophen, Microcrystalline Cellulose, Crospovidone, Silicon Dioxide, Magnesium Stearate and Polyvinyl Pyyrolidone in a mixer and compressed into a third release layer to form an immediate release layer.

In another embodiment of this invention, a dosage form comprising faxeladol and acetaminophen, a third layer is formed by mixing acetaminophen, Microcrystalline Cellulose, Crospovidone, Silicon Dioxide, Magnesium Stearate and Polyvinyl Pyyrolidone in a mixer and compressed into a third release layer to form an immediate release layer.

### Fourth Release Inert Layer (Optional);

The pharmaceutical dosage form of this invention may optionally comprise an inert layer. The inert layer constituents are easily discerned from the art. The inert layer devoid of any active agent, for example, may comprise of microcrystalline cellulose, magnesium stearate, Eudragit L100, and Poly Pyrrolidone. The inert layer is prepared by simple compression techniques known in the art and is used along with first, second and optionally third release layers to form a core as described below.

### Manufacturing Process: Core

The compositions for example, the first release layer, second release, a third release layer (which is optional) and an inert layer compositions are granulated, the granules of the individual layers are compressed to form a tablet using a rotary compression. When the compositions are processed by direct compression, the blends of the compositions respectively, may be compressed using a rotary press. The order of release layers is immaterial. For Example; in one embodiment of the invention, the first release layer comprising at least opioid dispersed in a slow-release matrix comprising cross-linked high amylose starch is compressed over a second release layer comprising acetaminophen dispersed in a slow-release matrix which in turn compressed over the inert layer. In another embodiment of this invention, the first release layer comprising at least one opioid dispersed in a slow-release matrix comprising cross-linked high amylose starch, is compressed below a second release layer comprising acetaminophen dispersed in a second-release matrix but above an inert layer. In another embodiment of this invention, the first release layer comprising at least one opioid dispersed in a slow-release matrix comprising cross-linked high amylose starch, is compressed over a second release layer comprising acetaminophen dispersed in a second-release matrix which in turn compressed over a third release layer, that optionally comprises either an opioid or acetaminophen or both and all three layers are compressed over an inert layer.

In another embodiment of the invention, the first release layer comprising at least opioid and acetaminophen dispersed in a slow-release matrix comprising cross-linked high amylose starch is compressed over a second release layer comprising acetaminophen and opioid dispersed in a slow-release matrix which in turn compressed over the inert layer.

### Manufacturing Process: Coat

The coating of the core is carried out by techniques known in the art. The coating solution, according to respective formulation tables, was prepared using known emulsion polymerization techniques.

For Example, in one embodiment of the present invention, the coating solution is prepared by dissolving the pore forming agent in water and adding the dispersion of the water insoluble polymer to it, and then mixing the two together until the water soluble compound is dissolved in the aqueous dispersion. The coating composes solid content ranging from about 5% to about 25% w/w, preferably from about 10% to about 20%, more preferably from about 10% to about 15% w/w. The coating may be a film that may include water insoluble polymers such as ethyl cellulose, cellulose acetate, polyvinyl acetate, nitrocellulose, butadiene styrene copolymers, and water insoluble methacrylate copolymers. In some embodiments, Eudragit RS100, Eudragit RS PO, Eudragit RS 30D and Eudragit RS 12.5 may be used. The polymers that are insoluble below a pH of about 4.0 but soluble at pH above 7.0 are also used in another embodiment of invention. Such polymers include Eudragit L 100, Eudragit L 12.5, Eudragit 12.5 P, Eudragit L 30 D-55, Eudragit L 100-55, Eastacryl 30 D, Kollicoat MAE 30 D and Kollicoat MAE 30 D, cellulose acetate phthalate, hydroxypropyl methyl cellulose acetate succinate and the like and mixture thereof. Aqueous Ethyl cellulose in a dispersion form is used in a preferred embodiment

### Manufacturing Process: Membrane Pouch

Poly Vinyl Alcohol (EMD EMPROVE® Ph. Eur. USP) 10 g, water 90 mg and Glycerol 3.1 g were mixed and heated to 95′ c. The hot polyvinyl solution was cooled to room temperature and spread on a glass plate to form a thin soft flexible film of about 100 μm thickness was formed. The film was cut suitably to prepare pouch or a sachet of appropriate size depending on the size of the solid dosage form. The pouch was used encapsulate the coated compressed core.

### Example 7

A gastro-retentive dosage form comprising 20 mg Oxycodone and 650 mg of Acetaminophen was prepared according to the formula in Table 7 as per the manufacturing described above.

TABLE 7

| | mg | percent |
|---|---|---|
| FIRST RELEASE LAYER | | |
| Acetaminophen | 390 | 54.3 |
| Oxycodone | 10 | 1.4 |
| Microcrystalline Cellulose | 18 | 2.5 |
| Starch | 33 | 4.6 |
| Cross Linked Amylose | 145 | 20.2 |
| Sodium Stearyl Fumarate | 11 | 1.5 |
| Hydroxypropylmethyl Cellulose | 43 | 6.0 |
| Colloidal Silicon Dioxide | 3.5 | 0.5 |
| Total Weight | 653.5 | 91.0 |
| SECOND RELEASE LAYER | | |
| Acetaminophen | 260 | 77.9 |
| Oxycodone | 10 | 3.0 |
| Microcrystalline Cellulose | 7.25 | 2.2 |
| Croscarmellose Sodium | 6.15 | 1.8 |
| Sodium Stearyl Fumarate | 4.62 | 1.4 |
| Starch | 29 | 8.7 |
| Colloidal Silicon Dioxide | 1.5 | 0.4 |
| FD & C Yellow 6 | 0.1 | 0.0 |
| Total Second Release Layer | 318.62 | 95.5 |
| Total Tablet Weight | 972.12 | |

### Example 8

A gastro-retentive dosage form comprising 50 mg Tapentadol and 300 mg of Acetaminophen was prepared according to the formula in Table 8 as per the manufacturing described above.

TABLE 8

| | mg | percent |
|---|---|---|
| CORE | | |
| First Release Layer | | |
| Tapentadol | 25 | 35.67 |
| Hydrogenated Vegetable Oil | 0.7 | 1.00 |
| Silica | 0.18 | 0.26 |
| Cross Linked Amylose | 43.5 | 62.07 |
| Magnesium Stearate | 0.7 | 1.00 |
| Total First Release Layer | 70.08 | 100.00 |
| Second Release Layer | | |
| Tapentadol | 25 | 10.87 |
| Hydrogenated Vegetable Oil | 2.6 | 1.13 |
| Silica | 0.5 | 0.22 |
| Magnesium Stearate | 1.3 | 0.57 |
| Kollidon SR ® | 133.7 | 58.13 |
| Xanthan Gum | 66.9 | 29.09 |
| Total Second Release Layer | 230 | 100.00 |
| Third Release Layer | | |
| Acetaminophen | 300 | 73.39 |
| Microcrystalline Cellulose | 50 | 12.23 |
| Crospovidone | 40 | 9.79 |
| Colloidal Silicon Dioxide | 5.5 | 1.35 |
| Magnesium Stearate | 2.5 | 0.61 |
| Polyvinyl Pyrrolidone | 10 | 2.45 |
| Talc | 0.75 | 0.18 |
| Total Third Layer | 408.75 | 100.00 |
| Inert Layer | | |
| Microcrystalline Cellulose | 75 | 40.98 |
| Eudragit L100 | 50 | 27.32 |
| Polyvinyl Pyrrolidone | 53 | 28.96 |
| Magnesium Stearate | 2.5 | 1.37 |
| Talc | 2.5 | 1.37 |
| Total Inert Layer | 183 | 100.00 |
| TOTAL CORE | 300.08 | |
| COAT | | |
| Ethyl Cellulose | 20 | 25.56 |
| Sodium Lauryl Sulfate | 0.75 | 0.96 |
| Aqucoat ECD | 50 | 63.90 |
| Cetyl Alcohol | 1.5 | 1.92 |
| Dibutyl Sebacate | 5 | 6.39 |
| Triethyl Citrate | 1 | 1.28 |
| Water | | |
| TOTAL COAT | 78.25 | 100.00 |
| TOTAL TABLET | 1087.16 | |

### Example 9

A gastro-retentive dosage form comprising 100 mg Tapentadol and 600 mg of Acetaminophen was prepared according to the formula in Table 9 as per the manufacturing described above.

TABLE 9

| | mg | percent |
|---|---|---|
| CORE | | |
| First Release Layer | | |
| Tapentadol | 50 | 35.70 |
| Hydrogenated Vegetable Oil | 1.35 | 0.96 |
| Silica | 0.36 | 0.26 |
| Cross Linked Amylose | 87 | 62.12 |
| Magnesium Stearate | 1.35 | 0.96 |
| Total First Release Layer | 140.06 | 100.00 |
| Second Release Layer | | |
| Tapentadol | 50 | 8.33 |
| Acetaminophen | 300 | 50.00 |
| Hydrogenated Vegetable Oil | 3.6 | 0.60 |
| Silica | 0.7 | 0.12 |
| Magnesium Stearate | 1.8 | 0.30 |
| Kollidon SR ® | 162.5 | 27.08 |
| Xanthan Gum | 81.4 | 13.57 |
| Total Second Release Layer | 600 | 100.00 |
| Third Release Layer | | |
| Acetaminophen | 300 | 73.39 |
| Microcrystalline Cellulose | 50 | |
| Crospovidone | 40 | 9.79 |
| Colloidal Silicon Dioxide | 5.5 | 1.35 |
| Magnesium Stearate | 2.5 | 0.61 |
| Polyvinyl Pyrrolidone | 10 | 2.45 |
| Talc | 0.75 | 0.18 |
| Total Third Layer | 408.75 | 87.77 |
| Inert Layer | | |
| Microcrystalline Cellulose | 133 | 58.33 |
| Eudragit L100 | 45 | 19.74 |
| Polyvinyl Pyrrolidone | 45 | 19.74 |
| Magnesium Stearate | 2.5 | 1.10 |
| Talc | 2.5 | 1.10 |
| Total Inert Layer | 228 | 100.00 |
| TOTAL CORE | 740.06 | |
| COAT | | |
| Ethyl Cellulose | 20 | 25.56 |
| Sodium Lauryl Sulfate | 0.75 | 0.96 |
| Aqucoat ECD | 50 | 63.90 |
| Cetyl Alcohol | 1.5 | 1.92 |
| Dibutyl Sebacate | 5 | 6.39 |
| Triethyl Citrate | 1 | 1.28 |
| Water | | |
| TOTAL COAT | 78.25 | 100.00 |
| TOTAL TABLET | 1315 | |

### Example 10

A gastro-retentive dosage form comprising 4 mg Hydromorphone and 300 mg of Acetaminophen was prepared according to the formula in Table 10 as per the manufacturing described above.

TABLE 10

| | mg | percent |
|---|---|---|
| CORE | | |
| First Release Layer | | |
| Hydromorphone | 4 | 12.67 |
| Hydrogenated Vegetable Oil | 0.7 | 2.22 |
| Silica | 0.18 | 0.57 |
| Cross Linked Amylose | 26 | 82.33 |
| Magnesium Stearate | 0.7 | 2.22 |
| Total First Release Layer | 31.58 | 100.00 |
| Second Release Layer | | |
| Acetaminophen | 300 | 65.30 |
| Hydrogenated Vegetable Oil | 2.6 | 0.57 |
| Silica | 0.5 | 0.11 |
| Magnesium Stearate | 1.3 | 0.28 |
| Kollidon SR ® | 100 | 21.77 |
| Xanthan Gum | 55 | 11.97 |
| Total Second Release Layer | 459.4 | 100.00 |
| Third Release Layer | | |
| Microcrystalline Cellulose | 50 | 47.08 |
| Crospovidone | 40 | 37.66 |
| Colloidal Silicon Dioxide | 3.2 | 3.01 |
| Magnesium Stearate | 2 | 1.88 |
| Polyvinyl Pyrrolidone | 10 | 9.42 |
| Talc | 1 | 0.94 |
| Total Third Layer | 106.2 | 100.00 |
| Inert Layer | | |
| Microcrystalline Cellulose | 55 | 42.97 |
| Eudragit L100 | 35 | 27.34 |
| Polyvinyl Pyrrolidone | 33 | 25.78 |
| Magnesium Stearate | 2.5 | 1.95 |
| Talc | 2.5 | 1.95 |
| Total Inert Layer | 128 | 100.00 |
| TOTAL CORE | 490.98 | |
| COAT | | |
| Ethyl Cellulose | 18 | 28.46 |
| Sodium Lauryl Sulfate | 0.75 | 1.19 |
| Aqucoat ECD | 37 | 58.50 |
| Cetyl Alcohol | 1.5 | 2.37 |
| Dibutyl Sebacate | 5 | 7.91 |
| Triethyl Citrate | 1 | 1.58 |
| Water | | |
| TOTAL COAT | 63.25 | 100.00 |
| TOTAL TABLET | 1151.41 | |

### Example 11

A gastro-retentive dosage form comprising 100 mg Tramadol and 600 mg of Acetaminophen was prepared according to the formula in Table 11 as per the manufacturing described above.

TABLE 11

| | mg | percent |
|---|---|---|
| CORE | | |
| First Release Layer | | |
| Tramadol | 100 | 52.53 |
| Hydrogenated Vegetable Oil | 0.7 | 0.37 |
| Silica | 0.18 | 0.09 |
| Cross Linked Amylose | 88 | 46.22 |
| Magnesium Stearate | 1.5 | 0.79 |
| Total First Release Layer | 190.38 | 100.00 |
| Second Release Layer | | |
| Acetaminophen | 600 | 75.59 |
| Microcrystalline Cellulose | 130 | 16.38 |
| Crospovidone | 40 | 5.04 |
| Colloidal Silicon Dioxide | 5.5 | 0.69 |
| Magnesium Stearate | 2.5 | 0.31 |
| Polyvinyl Pyrrolidone | 15 | 1.89 |
| Talc | 0.75 | 0.09 |
| Total Second Release Layer | 793.75 | 24.41 |
| Membrane Pouch | | |
| Polyvinyl Alcohol | 275 | 85.67 |
| Glycerol | 46 | 14.33 |
| Total Inert Layer | 321 | 100.00 |
| TOTAL CORE | 1305.13 | |
| COAT | | |
| Ethyl Cellulose | 20 | 19.18 |
| Sodium Lauryl Sulfate | 0.75 | 0.72 |
| Aquacoat ECD | 75 | 71.94 |
| Cetyl Alcohol | 1.5 | 1.44 |
| Dibutyl Sebacate | 6 | 5.76 |
| Triethyl Citrate | 1 | 0.96 |
| Water | | |
| TOTAL COAT | 104.25 | 100.00 |
| TOTAL TABLET | 1409.38 | |

### Example 12

A gastro-retentive dosage form comprising 50 mg Tapentadol and 300 mg of Acetaminophen was prepared according to the formula in Table 12 as per the manufacturing described above.

TABLE 12

| | mg | percent |
|---|---|---|
| CORE | | |
| First Release Layer | | |
| Tapentadol | 50 | 30.93 |
| Hydrogenated Vegetable Oil | 1.5 | 0.93 |
| Silica | 0.18 | 0.11 |
| Cross Linked Amylose | 108 | 66.80 |
| Magnesium Stearate | 2 | 1.24 |
| Total First Release Layer | 161.68 | 100.00 |
| Second Release Layer | | |
| Acetaminophen | 300 | 143.71 |
| Microcrystalline Cellulose | 130 | 62.28 |
| Crospovidone | 50 | 23.95 |
| Colloidal Silicon Dioxide | 6.8 | 3.26 |
| Magnesium Stearate | 3.2 | 1.53 |
| Polyvinyl Pyrrolidone | 18 | 8.62 |
| Talc | 0.75 | 0.36 |
| Total Second Release Layer | 208.75 | 100.00 |
| Membrane Pouch | | |
| Polyvinyl Alcohol | 360 | 84.51 |
| Glycerol | 66 | 15.49 |
| Total Inert Layer | 426 | 100.00 |
| TOTAL CORE | 796.43 | |
| COAT | | |
| Ethyl Cellulose | 20 | 19.37 |
| Sodium Lauryl Sulfate | 0.75 | 0.73 |
| Aquacoat ECD | 75 | 72.64 |
| Cetyl Alcohol | 1.5 | 1.45 |
| Dibutyl Sebacate | 5 | 4.84 |
| Triethyl Citrate | 1 | 0.97 |
| Water | | |
| TOTAL COAT | 103.25 | 100.00 |
| TOTAL TABLET | 899.68 | |

### Example 13

A gastro-retentive dosage form comprising 5 mg Hydrocodone and 500 mg of Acetaminophen was prepared according to the formula in Table 13 as per the manufacturing described above.

TABLE 13

| | mg | percent |
|---|---|---|
| CORE | | |
| First Release Layer | | |
| Hydrocodone | 5 | 9.98 |
| Hydrogenated Vegetable Oil | 0.7 | 1.40 |
| Silica | 0.18 | 0.36 |
| Cross Linked Amylose | 43.5 | 86.86 |
| Magnesium Stearate | 0.7 | 1.40 |
| Total First Release Layer | 50.08 | 100.00 |
| Second Release Layer | | |
| Acetaminophen | 500 | 70.92 |
| Hydrogenated Vegetable Oil | 2.6 | 0.37 |
| Silica | 0.5 | 0.07 |
| Magnesium Stearate | 1.3 | 0.18 |
| Kollidon SR ® | 133.7 | 18.96 |
| Xanthan Gum | 66.9 | 9.49 |
| Total Second Release Layer | 705 | 100.00 |
| Third Release Layer | | |
| Microcrystalline Cellulose | 60 | 49.69 |
| Crospovidone | 35 | 28.99 |
| Colloidal Silicon Dioxide | 7.5 | 6.21 |
| Magnesium Stearate | 2.5 | 2.07 |
| Polyvinyl Pyrrolidone | 15 | 12.42 |
| Talc | 0.75 | 0.62 |
| Total Third Layer | 120.75 | 100.00 |
| Membrane Pouch | | |
| Polyvinyl Alcohol | 215 | 83.01 |
| Glycerol | 44 | 16.99 |
| Total Covering | 259 | 100.00 |
| TOTAL CORE | 1134.83 | |
| COAT | | |
| Ethyl Cellulose | 20 | 19.37 |
| Sodium Lauryl Sulfate | 0.75 | 0.73 |
| Aquacoat ECD | 75 | 72.64 |
| Cetyl Alcohol | 1.5 | 1.45 |
| Dibutyl Sebacate | 5 | 4.84 |
| Triethyl Citrate | 1 | 0.97 |
| Water | | |
| TOTAL COAT | 103.25 | 100.00 |
| TOTAL TABLET | 1238.08 | |

### Example 14

A gastro-retentive dosage form comprising 50 mg Axomadol and 300 mg of Acetaminophen was prepared according to the formula in Table 14 as per the manufacturing described above.

TABLE 14

| | mg | percent |
|---|---|---|
| CORE | | |
| First Release Layer | | |
| Axomadol | 50 | 49.71 |
| Hydrogenated Vegetable Oil | 0.7 | 0.70 |
| Silica | 0.18 | 0.18 |
| Cross Linked Amylose | 49 | 48.72 |
| Magnesium Stearate | 0.7 | 0.70 |
| Total First Release Layer | 100.58 | 100.00 |
| Second Release Layer | | |
| Acetaminophen | 300 | 65.65 |
| Microcrystalline Cellulose | 100 | 21.88 |
| Crospovidone | 40 | 8.75 |
| Colloidal Silicon Dioxide | 4.2 | 0.92 |
| Magnesium Stearate | 2 | 0.44 |
| Polyvinyl Pyrrolidone | 10 | 2.19 |
| Talc | 0.75 | 0.16 |
| Total Second Release Layer | 456.95 | 34.35 |
| Third Release Layer | | |
| Microcrystalline Cellulose | 66 | 49.13 |
| Crospovidone | 45 | 33.49 |
| Colloidal Silicon Dioxide | 6.1 | 4.54 |
| Magnesium Stearate | 1.5 | 1.12 |
| Polyvinyl Pyrrolidone | 15 | 11.16 |
| Talc | 0.75 | 0.56 |
| Total Third Layer | 134.35 | 100.00 |
| Membrane Pouch | | |
| Polyvinyl Alcohol | 199 | 83.61 |
| Glycerol | 39 | 16.39 |
| Total Covering | 238 | 100.00 |
| TOTAL CORE | 929.88 | |
| COAT | | |
| Ethyl Cellulose | 20 | 19.37 |
| Sodium Lauryl Sulfate | 0.75 | 0.73 |
| Aquacoat ECD | 75 | 72.64 |
| Cetyl Alcohol | 1.5 | 1.45 |
| Dibutyl Sebacate | 5 | 4.84 |
| Triethyl Citrate | 1 | 0.97 |
| Water | | |
| TOTAL COAT | 103.25 | 100.00 |
| TOTAL TABLET | 1033.13 | |

### Example 15

A gastro-retentive dosage form comprising 60 mg morphine and 300 mg of Acetaminophen was prepared according to the formula in Table 15 as per the manufacturing described above.

TABLE 15

| | mg | percent |
|---|---|---|
| CORE | | |
| First Release Layer | | |
| Morphine | 60 | 62.12 |
| Hydrogenated Vegetable Oil | 0.7 | 0.72 |
| Silica | 0.18 | 0.19 |
| Cross Linked Amylose | 35 | 36.24 |
| Magnesium Stearate | 0.7 | 0.72 |
| Total First Release Layer | 96.58 | 100.00 |
| Second Release Layer | | |
| Acetaminophen | 300 | 62.75 |
| Microcrystalline Cellulose | 112 | 23.43 |
| Crospovidone | 44 | 9.20 |
| Colloidal Silicon Dioxide | 4.1 | 0.86 |
| Magnesium Stearate | 2.2 | 0.46 |
| Polyvinyl Pyrrolidone | 15 | 3.14 |
| Talc | 0.75 | 0.16 |
| Total Second Release Layer | 478.05 | 37.25 |
| Third Release Layer | | |
| Microcrystalline Cellulose | 66 | 49.87 |
| Crospovidone | 45 | 34.00 |
| Colloidal Silicon Dioxide | 6.1 | 4.61 |
| Magnesium Stearate | 2.5 | 1.89 |
| Polyvinyl Pyrrolidone | 12 | 9.07 |
| Talc | 0.75 | 0.57 |
| Total Third Layer | 132.35 | 100.00 |
| Membrane Pouch | | |
| Polyvinyl Alcohol | 223 | 82.90 |
| Glycerol | 46 | 17.10 |
| Total Covering | 269 | 100.00 |
| TOTAL CORE | 975.98 | |
| COAT | | |
| Ethyl Cellulose | 22 | 20.51 |
| Sodium Lauryl Sulfate | 0.75 | 0.70 |
| Aquacoat ECD | 77 | 71.79 |
| Cetyl Alcohol | 1.5 | 1.40 |
| Dibutyl Sebacate | 5 | 4.66 |
| Triethyl Citrate | 1 | 0.93 |
| Water | | |
| TOTAL COAT | 107.25 | 100.00 |
| TOTAL TABLET | 1083.23 | |

### Example 16

A gastro-retentive dosage form comprising 50 mg Tapentadol and 300 mg of Acetaminophen was prepared according to the formula in Table 16 as per the manufacturing described above.

TABLE 16

| | mg | percent |
|---|---|---|
| CORE | | |
| First Release Layer | | |
| Acetaminophen | 300 | 72.55 |
| Hydrogenated Vegetable Oil | 1 | 0.24 |
| Silica | 0.5 | 0.12 |
| Cross Linked Amylose | 110 | 26.60 |
| Magnesium Stearate | 2 | 0.48 |
| Total First Release Layer | 413.5 | 100.00 |
| Second Release Layer | | |
| Tapentadol | 50 | 27.56 |
| Microcrystalline Cellulose | 110 | 60.62 |
| Crospovidone | 45 | 24.80 |
| Colloidal Silicon Dioxide | 6 | 3.31 |
| Magnesium Stearate | 2.7 | 1.49 |
| Polyvinyl Pyrrolidone | 17 | 9.37 |
| Talc | 0.75 | 0.41 |
| Total Second Release Layer | 181.45 | 100.00 |
| Third Release Layer | | |
| Microcrystalline Cellulose | 66 | 49.72 |
| Crospovidone | 45 | 33.90 |
| Colloidal Silicon Dioxide | 5.5 | 4.14 |
| Magnesium Stearate | 2.5 | 1.88 |
| Polyvinyl Pyrrolidone | 13 | 9.79 |
| Talc | 0.75 | 0.56 |
| Total Third Layer | 132.75 | 100.00 |
| Membrane Pouch | | |
| Polyvinyl Alcohol | 197 | 82.43 |
| Glycerol | 42 | 17.57 |
| Total Covering | 239 | 100.00 |
| TOTAL CORE | 966.7 | |
| COAT | | |
| Ethyl Cellulose | 23 | 23.65 |
| Sodium Lauryl Sulfate | 0.75 | 0.77 |
| Aquacoat ECD | 65 | 66.84 |
| Cetyl Alcohol | 1.5 | 1.54 |
| Dibutyl Sebacate | 6 | 6.17 |
| Triethyl Citrate | 1 | 1.03 |
| Water | | |
| TOTAL COAT | 97.25 | 100.00 |
| TOTAL TABLET | 1063.95 | |

### Example 17

A gastro-retentive bilayer dosage form comprising 100 mg Tapentadol and 600 mg of Acetaminophen was prepared according to the formula in Table 17 as per the manufacturing described above.

TABLE 17

| | mg | percent |
|---|---|---|
| CORE | | |
| First Release Layer | | |
| Tapentadol | 75 | 9.26 |
| Acetaminophen | 450 | |
| Starch | 45 | |
| Microcrystalline Cellulose | 35 | 4.32 |
| HPMC | 90 | 11.11 |
| Cross Linked Amylose | 80 | 9.88 |
| Crospovidone | 31 | |
| Silica | 3.1 | |
| Magnesium Stearate | 0.7 | 0.09 |
| Total First Release Layer | 809.8 | 34.66 |
| Second Release Layer | | |
| Acetaminophen | 150 | 79.53 |
| Starch | 22 | 11.66 |
| Microcrystalline Cellulose | 7.5 | 3.98 |
| Croscarmellose Sodium | 5.1 | 2.70 |
| Sodium Stearyl Fumarate | 2 | 1.06 |
| Colloidal Silicon Dioxide | 1.5 | |
| FD & C | 0.5 | 0.27 |
| Total Second Release Layer | 188.6 | 99.20 |
| COAT | | |
| Ethyl Cellulose | 20 | 19.37 |
| Sodium Lauryl Sulfate | 0.75 | 0.73 |
| Aquacoat ECD | 75 | 72.64 |
| Cetyl Alcohol | 1.5 | 1.45 |
| Dibutyl Sebacate | 5 | 4.84 |
| Triethyl Citrate | 1 | 0.97 |
| Water | | |
| TOTAL COAT | 103.25 | 100.00 |
| TOTAL TABLET | 1101.65 | |

### Example 18

A gastro-retentive abuse deterrent dosage form comprising 50 mg Tapentadol, 300 mg of Acetaminophen and 5 mg Naloxone was prepared according to the formula in Table 18 as per the manufacturing described above.

TABLE 18

| | mg | percent |
|---|---|---|
| CORE | | |
| First Release Layer | | |
| Tapentadol | 50 | 20.48 |
| Polyethylene Oxide | 150 | 61.43 |
| Hydroxypropylmethylcellulose | 0.5 | 0.20 |
| Magnesium Stearate | 0.18 | 0.07 |
| Ethyl Alcohol | 43.5 | 17.81 |
| Total First Release Layer | 244.18 | 100.00 |
| Second Release Layer | | |
| Naloxone | 5 | 4.08 |
| Polyethylene Oxide | 35 | 28.57 |
| Ferric Oxide | 0.1 | 0.08 |
| Hydroxypropylmethylcellulose | 0.5 | 0.41 |
| Sodium Chloride | 15 | 12.24 |
| Ethyl Alcohol* | 66.9 | 54.61 |
| Total Second Release Layer | 122.5 | 100.00 |
| Semi permeable Coat | | |
| Cellulose Acetate | 80 | 95.24 |
| Polyethylene Glycol | 4 | 4.76 |
| Acetone:Water (95:5)* | | 0.00 |
| Total Coat | 84 | 100.00 |
| TOTAL CORE | 450.68 | |
| Immediate Release Layer | | |
| Acetaminophen | 300 | 58.59 |
| PVP | 45 | 8.79 |
| Lactose, Hydrogen Dried | 125 | 24.41 |
| Magnesium Stearate | 1.5 | 0.29 |
| Aquacoat ECD (as 30% W/W | 16.5 | 3.22 |
| suspension) | | |
| Methocel | 16.5 | 3.22 |
| Triethyl Citrate | 7.5 | 1.46 |
| Water* | | |
| Total Immediate Release Layer | 512 | 100.00 |
| COAT | | |
| Ethyl Cellulose | 20 | 25.56 |
| Sodium Lauryl Sulfate | 0.75 | 0.96 |
| Aqucoat ECD | 50 | 63.90 |
| Cetyl Alcohol | 1.5 | 1.92 |
| Dibutyl Sebacate | 5 | 6.39 |
| Triethyl Citrate | 1 | 1.28 |
| Water | | |
| TOTAL COAT | 78.25 | 100.00 |
| TOTAL TABLET | 1040.93 | |

### Manufacturing Process;

The tapentadol and antagonist core was prepared according procedure reported in U.S. Pat. No. 5,866,164. For Example; the requisite quantity of Tapentadol, Polyethylene Oxide and Hydroxypropylmethylcellulose are all nicely blended in roll mill and dried. Denatured ethanol was added to the dry blend and dried at room temperature. The dried mass was pressed through a screener and dried again for 48 hours. Lubricant magnesium stearate is added to the dry mass and homogenized. Polyethylene Oxide, HPMC, Sodium Chloride, Ferric Oxide were mixed with denatured alcohol and blended to attain homogeneity. The homogenized mass was passed through a screener and dried for 48 hours. Lubricant was added during the granulation. Tapentadol and Naloxone were compressed (2; 1 ratio) into a bilayer tablet core. The tablets were coated with a semi permeable membrane comprising cellulose acetate and PEG, in a coater and a passage was drilled according to standard procedures. The acetaminophen layer constituents were mixed and compressed over semi permeable membrane coated tablets. An optional coating was applied over the acetaminophen layered semi permeable membrane coated tapentadol and Naloxone tablets.

### Dissolution Studies;

The present inventors found that in order to achieve a slow release profile of tapentadol in a combination comprising an opioid (tapentadol) and acetaminophen, over at least a twelve hour period following the administration of drug, the in vitro release rate preferably corresponds to the following rate of tapentadol released; between 0 and 50% tapentadol released after 1 hour; between 0 and 75% tapentadol released after 2 hours; between 3 and 95% tapentadol released after 4 hours; between 10 and 100% tapentadol released after 8 hours; between 20 and 100% tapentadol released after 12 hours; between 30 and 100% tapentadol released after 16 hours; between 50 and 100% tapentadol released after 24 hours; and greater than 80% tapentadol released after 36 hours, by weight.

The acetaminophen release corresponds to the following: between 10% and 50% acetaminophen released after 1 hour; between 30 and 90% acetaminophen released after 4 hours; between 50% and 90% acetaminophen released after 8 hours; not less than 90% acetaminophen is released after 12 hours.

Another preferred preparation especially suited for twice-a-day dosing has an in vitro release rate corresponding to the following % rate of tapentadol released: between 20 and 50% tapentadol released after 1 hour; between 40 and 75% tapentadol released after 2 hours; between 60 and 95% tapentadol released after 4 hours; between 80 and 100% tapentadol released after 8 hours; between 90 and 100% tapentadol released after 12 hours; by weight

Yet another preferred preparation particularly suited for once-a-day dosing has an in-vitro release rate corresponding to the following % rate of tapentadol released: between 0 and 50% tapentadol released after 1 hour; between 0 and 75% tapentadol released after 2 hours; between 10 and 95% tapentadol released after 4 hours; between 35 and 100% tapentadol released after 8 hours; between 55 and 100% tapentadol released after 12 hours; between 70 and 100% tapentadol released after 16 hours; greater than 90% tapentadol released after 24 hours, by weight

A still further preferred preparation in accordance with the invention also particularly suited for once-a-day dosing has an in vitro release rate corresponding to the following % rate if tapentadol released; between 0 and 30% tapentadol released after 1 hour; between 0 and 40% tapentadol released after 2 hours; between 3 and 55% tapentadol released after 4 hours; between 10 and 65% tapentadol released after 8 hours; between 20 and 75% tapentadol released after 12 hours; between 30 and 88% tapentadol released after 16 hours; between 50 and 100% tapentadol released after 24 hours, greater than 80% tapentadol released after 36 hours, by weight

More preferably a preparation for once-a-day dosing has an in vitro release rate substantially as follows: between 15 and 25% tapentadol released after 1 hour; between 25 and 35% tapentadol released after 2 hours; between 30 and 45% tapentadol released after 4 hours; between 40 and 60% tapentadol released after 8 hours; between 55 and 70% tapentadol released after 12 hours; between 60 and 75% tapentadol released after 16 hours; by weight

The dissolution profiles of Example 1 (Immediate Release tapentadol), Example 2 (Slow Release Core) and Example 3 (Slow release Dosage Form) are listed below;

TABLE 6

| Time | |
|---|---|
| | Dissolution Profile of Example 1 |
| | Example 1 |
| 0 | 0 |
| 2 | 0-100% |
| 4 | Greater Than 70% |
| | Dissolution Profile of Example 2 |
| | Example 2 |
| 0 | 0 |
| 2 | 0-10% |
| 4 | 10-40% |
| 6 | 40-70% |
| 8 | More than 60% |
| 12 | More than 70% |
| 16 | More than 80% |
| | Dissolution Profile of Example 3 |
| | Example 3 |
| 0 | 0 |
| 2 | 0-60% |
| 4 | 25-85% |
| 6 | 45-90% |
| 8 | More than 60% |
| 12 | More than 70% |
| 16 | More than 80% |

### Bioavailability Studies;

A total of 12 subjects were enrolled in the study to evaluate the pharmacokinetics/bioavailability of tapentadol and acetaminophen and all of them randomly received drugs as follows:

Fixed Dose: Example 3 (Tapentadol 50 mg+Acetaminophen 300 mg) administered once

Co-administered Dose: Example 2 (Tapentadol 50 mg)+Reference Example 6 (Acetaminophen 300 mg) administered once.

The study included two treatment phases wherein each phase was separated by washout period of 21 days. Subjects were randomized to receive one of the above two regimens as randomly assigned by Latin Square and each subject crossed to each regimen according to the randomization sequence until all subjects have received all two regimens (with twenty one week separating each regimen). Blood samples were centrifuged within 2 hours of collection and the plasma were separated and frozen at −10′ C or lower until assayed. HPLC Analysis was carried out using stand techniques known to the person skilled in art. Similarly Examples 1 and 6 were administered to patients after a wash out period and samples were analyzed.

Tables 7 and 8 show a summary of the pharmacokinetics parameters of tapentadol and acetaminophen when administered as fixed dose and as co-administered doses.

TABLE 7
Tapentadol Parameters

| | Tapentadol + | Tapentadol + |
|---|---|---|
| | Acetaminophen | Acetaminophen |
| Parameter | (Co-Administration) | (Fixed Dose) |
| tₘₐₓ hours | 1.10 (1.00-5.00) | 1.00 (1.00-6.00) |
| Cₘₐₓ ng/mL | 44.4 ± 22.4 | 42.5 ± 21.8 |
| AUCₜₑₛₜ ng · h/mL | 195.62 ± 42.4 | 194.33 ± 49.1.4 |
| AUC_{∞} ng · h/mL | 197.66 ± 59.2 | 197.82 ± 879.1 |
| t_{1/2} hours | 4.1 ± 0.4 | 4.0 ± 0.3 |

TABLE 8
Acetaminophen Parameters

| | Tapentadol + | Tapentadol + |
|---|---|---|
| | Acetaminophen | Acetaminophen |
| Parameter | (Co-Administration) | (Fixed Dose) |
| tₘₐₓ hours | 0.9 (0.6-1.1) | 0.92(0.7-1.3) |
| Cₘₐₓ ng/mL | 3998 ± 446.3 | 4098.78 ± 556.12 |
| AUCₜₑₛₜ ng · h/mL | 14456.4 ± 1125.2 | 13989.1 ± 1425.3 |
| AUC_{∞} ng · h/mL | 15128.47 ± 2587.78 | 15456.81 ± 2777.56 |
| t_{1/2} hours | 2.58 ± 0.56 | 2.49 ± 0.45 |

The data was analyzed for variance between the treatment for both tapentadol and acetaminophen parameters in fixed dose and co-administered doses. All 90% confidence interval for fixed dose and co-administered treatments ratios were included within 80%-125%, the variance accepted for bioequivalence. Please see Table 9

TABLE 9
Ratio

| | Fixed Dose/Co- | |
|---|---|---|
| | Administration | 90% CI |
| Tapentadol Parameters | | |
| Cₘₐₓ ng/mL | 95.11 | 86.22-111.4 |
| AUCₜₑₛₜ ng · h/mL | 99.22 | 95.33-109.98 |
| AUC_{∞} ng · h/mL | 100.2 | 95.22-109.44 |
| Acetaminophen | | |
| parameters | | |
| Cmax ng/mL | 98.11 | 90.33-110.3 |
| AUC test ng · h/mL | 96.3 | 93.33-107.55 |
| AUC ∞ ng · h/mL | 100.1 | 95.22-108.67 |

### Clinical Studies;

A human clinical trial was conducted to compare the efficacy and safety of a pharmaceutical composition comprising a therapeutically effective amount of slow release tapentadol and at least one form of acetaminophen at least one pharmaceutically acceptable excipient, prepared according to this invention with placebo and against slow release tapentadol and acetaminophen mono therapy. It was found very surprisingly that the pharmaceutical composition comprising a therapeutically effective amount of slow release tapentadol and at least one pharmaceutically acceptable excipient of present invention to have analgesic efficacy over the 12-hour dosing interval which is superior to the analgesic efficacy of mono therapy.

Knee Osteoarthritis, widely considered as the appropriate chronic pain model for studying the efficacy of pain medications, was chosen for the study that involved 98 patients in three arms. 6 patients dropped out and discontinued by investigators. The WOMAC questionnaire was administered to patients at each study visit and composite scores for each sub-scale and the global score were reported separately.

The WOMAC questionnaire is a statistically validated pain measurement indicator and The WOMAC (Western Ontario and McMaster Universities) function subscale is widely used in clinical trials of hip and knee osteoarthritis. Reducing the number of items of the subscale would enhance efficiency and compliance, particularly for use in clinical practice applications.

### Study Design:

The trial was designed as an Interventional, Randomized, Double-Blind, and Multi-Center and Parallel Assignment study for a period of 12 weeks (Day D₀-Day D₉₀). After randomization to Slow Release Tapentadol 50+Acetaminophen 300 mg (Example 12, BID), Slow Release Tapentadol 50 mg (Example 2, BID) or Acetaminophen 300 mg (Example 6 BID) or placebo, patient's dose was titrated to the fixed randomized dose and maintained for 12 weeks. At screening all pain medication and prohibited medications were discontinued prior to randomization in order to obtain unbiased baseline values for the efficacy variables and to ensure that patients had sufficiently severe OA. After baseline deadline, the eligible patients were randomly assigned to a treatment group and were titrated until an optimum dose was established (range 50-400 mg). Patients were then treated at the fixed optimum dose for rest of the duration of the trial of 12 weeks. In order to assess the non-inferiority of the investigative drug Example 3 and compared to Slow Release Tapentadol 50 mg (Example 2, BID) or Acetaminophen 300 mg (Example 6 BID) a 2-way ANCOVA was performed on the percentage change in the WOMAC pain sub-scale score from baseline to the last visit while adjusting for the baseline score.

### Study Management Summary;

Preliminary Baseline: Patients were screened as per inclusion and exclusion criteria and underwent analgesic washout on Day Dₒ and randomized to one of the treatment arms (Day D₀).

Titration; Patients were started with Investigative drug 50 mg Tapentadol and 300 mg Acetaminophen or Slow Release Tapentadol 50 mg or Acetaminophen 300 mg or Placebo with full blinding Day D₁. The patients were titrated to achieve optimum dosage Day D₁-D₄.

Observation and Maintenance: Patients received their respective randomized titrated optimum dosage for rest of the 12 weeks. (Day D₆-D₉₀).

### Inclusion Criteria:

Patients aged between 40-50 years (male or female) diagnosed with moderate to severe knee osteoarthritis with current pain.

Patients with a WOMAC (Western Ontario and McMaster University) Osteoarthritis index score of 150 nm at baseline.

Patients with ability to understand the oral and written communication.

### Exclusion Criteria:

Patients with other current arthritis diseases and those with history of failure or discontinuation (due to adverse events) of any other pain medication for osteoarthritis.

Patients with a history of major illness or those who were on monoamine oxidase inhibitors; tricyclic antidepressants and other tricyclic compounds; neuroleptics; selective serotonin reuptake inhibitors, within three months prior to the screening date,

Patients who were Pregnant or lactating or unwilling to utilize a medically approved method of contraception among those with childbearing potential.

Current or previous dependency on opioid or known substance abuse.

Effusion of greater than 15 upon physical examination,

Other minor criteria as deemed by investigator.

### Clinical Arms:

Slow Release Tapentadol 50 mg+Acetaminophen 300 mg, twice daily: Experimental Drug-Example 12 (N=15)

Slow Release Tapentadol 50 mg Twice daily 50 mg (BID): Example 2 (N=16)

Acetaminophen 300 mg Twice daily (BID); Example 6 (N=14)

Placebo: Placebo Comparator Drug: Placebo (N=15)

### Primary Outcome Measures

Percentage difference between Score at Baseline and at the end of the study (Day D₉₀) for WOMAC Pain Global Score and WOMAC Subscales; Pain Subscale, Stiffness Subscale and Physical Function Subscale,

### Secondary Outcome Measures

Incidence of adverse events that were typical of tapentadol (nausea, dizziness/vertigo, vomiting, somnolence, and constipation)

### Results

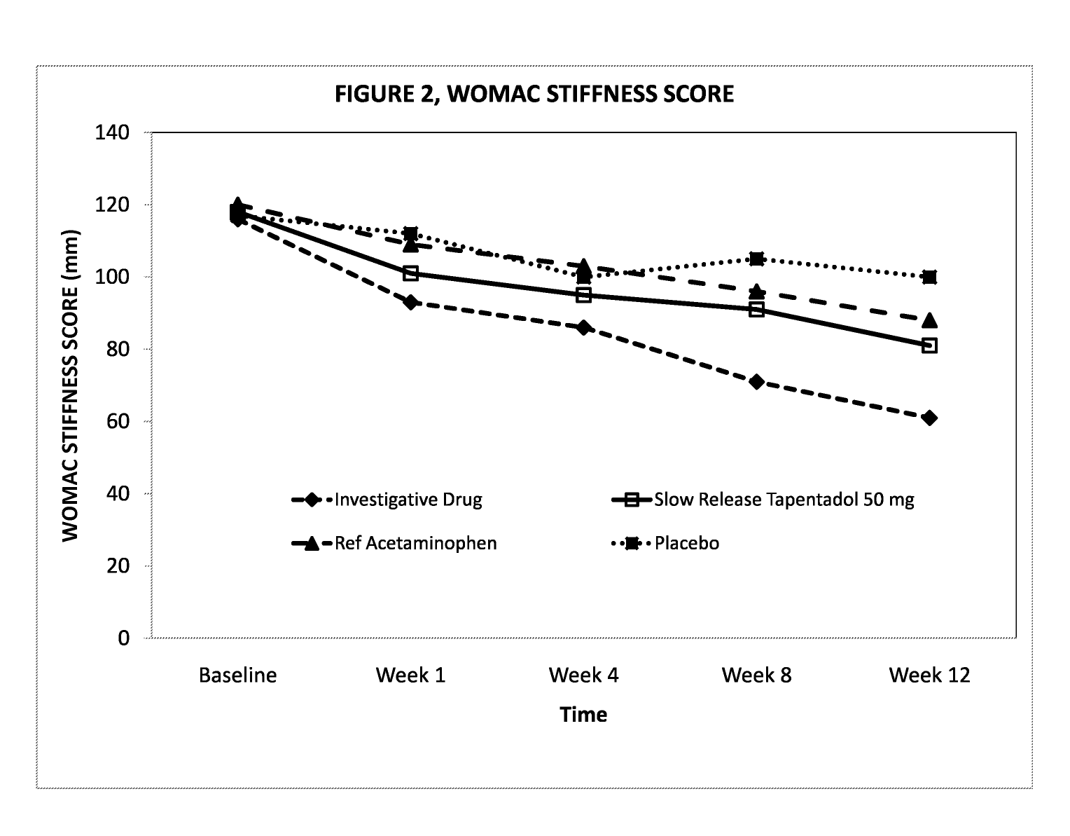

The results are FIGS. 2-5 where the WOMAC Global Scores and WOMAC Subscales are compared.

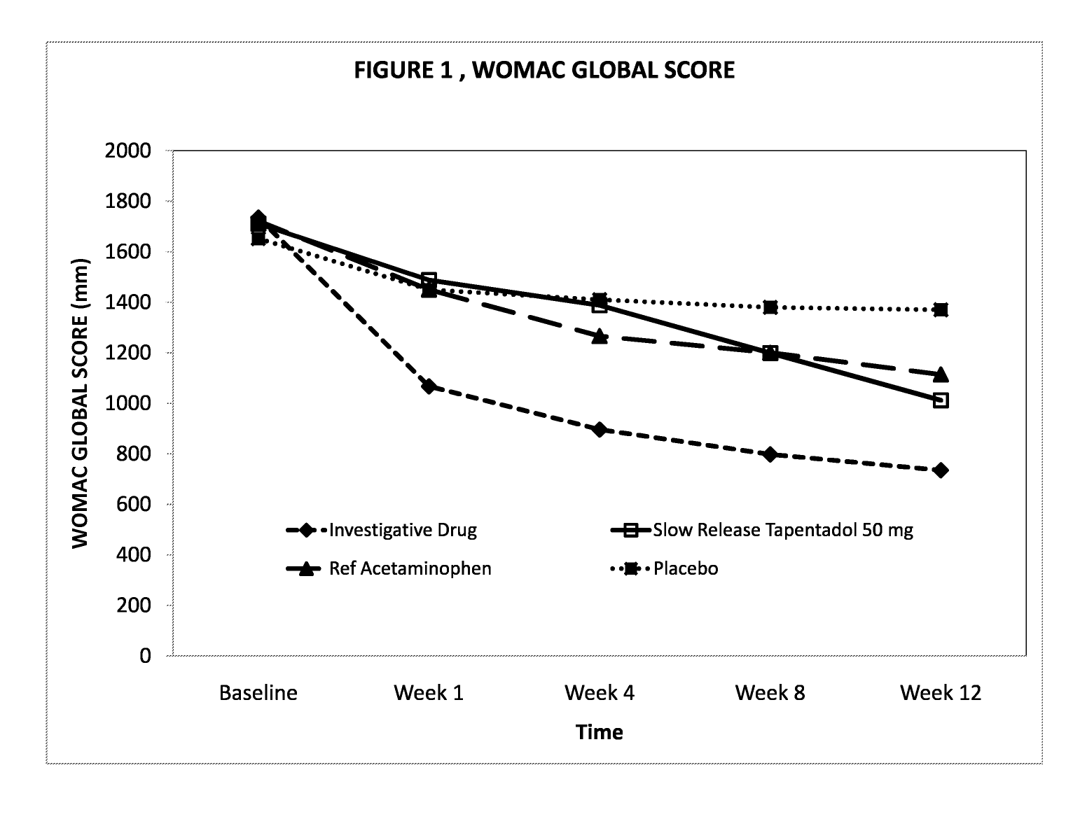

FIG. 1: The WOMAC Global Score from human clinical trial of Slow Release Tapentadol+Acetaminophen twice daily investigative drug against Slow Release Tapentadol two times daily and Acetaminophen two times daily reference drugs and Placebo.

FIG. 2: The WOMAC Subscale Stiffness Score from human clinical trial of Slow Release Tapentadol+Acetaminophen twice daily investigative drug against Slow Release Tapentadol two times daily and Acetaminophen two times daily reference drugs and Placebo.

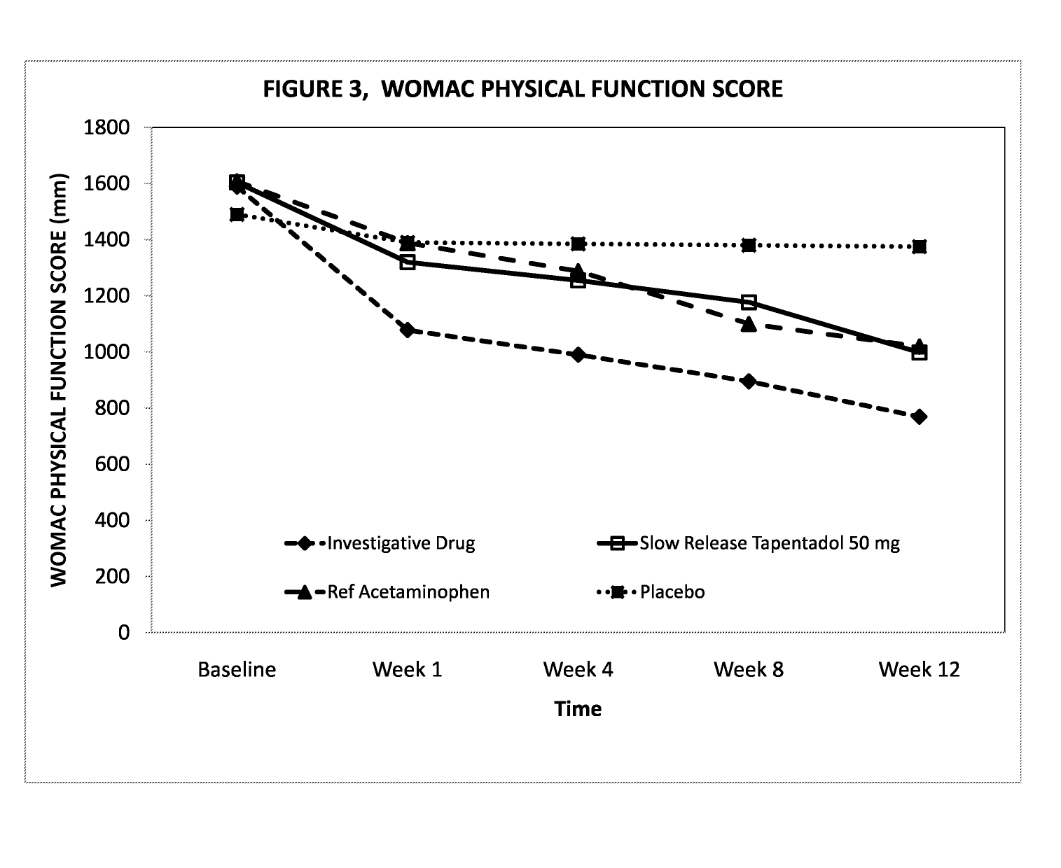

FIG. 3: The WOMAC Subscale Physical Function Score from human clinical trial of Slow Release Tapentadol+Acetaminophen twice daily investigative drug against Slow Release Tapentadol two times daily and Acetaminophen two times daily reference drugs and Placebo.

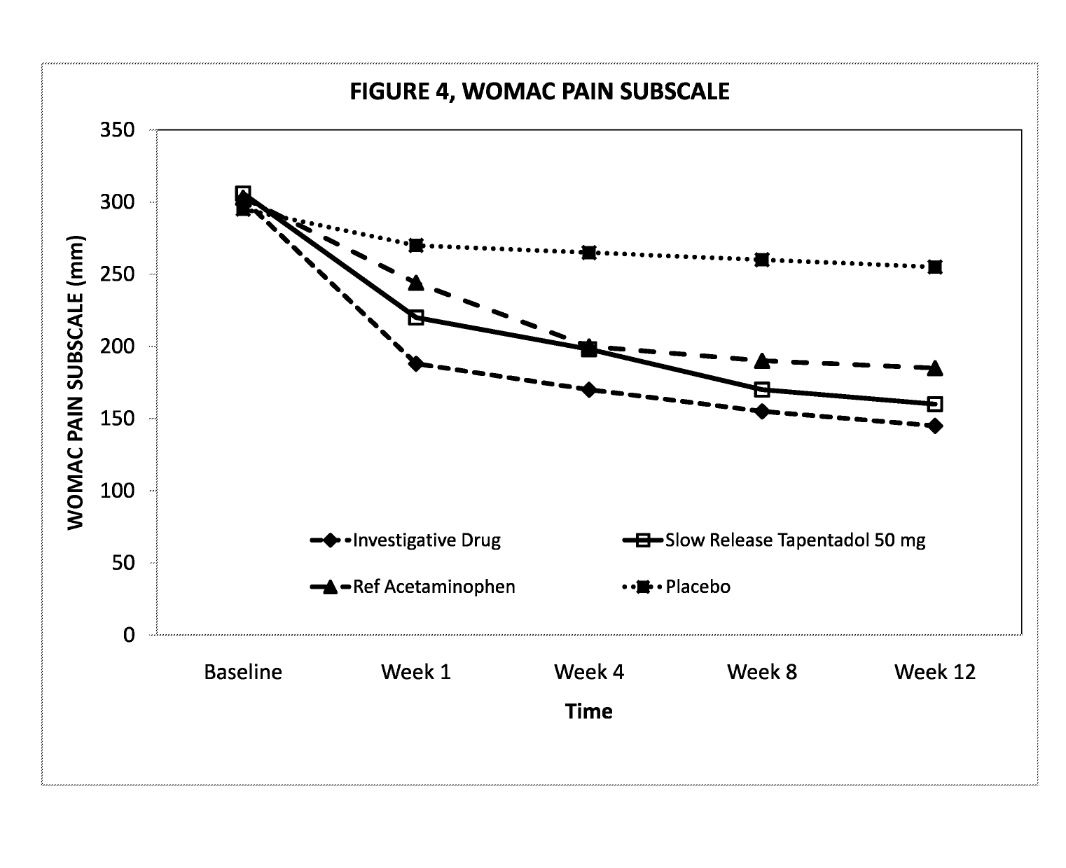

FIG. 4: The WOMAC Subscale Pain Score from human clinical trial of Slow Release Tapentadol+Acetaminophen twice daily investigative drug against Slow Release Tapentadol two times daily and Acetaminophen two times daily reference drugs and Placebo.

Thus, the treatment with an effective twice daily formulation of this invention is capable of rapid titration offers patients with chronic pain a significant advantage over mono therapy with individual drugs. The benefit of increased compliance and convenience due to a simplified dosing regimen is relevant to all patients and in particular to elderly patients, who often take multiple drugs and regimen, for which simplification of treatment regimens is a key treatment benefit and also provides as a safety issue. This, in combination with the fact that treatment with the inventive composition offers a clinically favorable adverse events profile in comparison of other tapentadol drugs supports the use of the drugs according instant invention as an analgesic treatment of choice for pain management.

In summary, this invention is related to the novel pharmaceutical composition comprising a therapeutically effective amount of slow release tapentadol and acetaminophen and at least one pharmaceutically acceptable excipient wherein the composition provides a clinical effect over 12 hours for treating pain.

Further embodiments (Emb) of the invention are:

### Emb-1

A pharmaceutical dosage form comprising a therapeutically effective amount of at least one opioid, acetaminophen and at least one pharmaceutically acceptable excipient wherein the said dosage form is retained in the stomach for at least four hours and is suitable for once daily or twice daily administration.

### Emb-2

A method of treating a disorder by administering a pharmaceutical dosage form comprising a therapeutically effective amount of at least one opioid, acetaminophen, and at least one pharmaceutically acceptable excipient wherein the said dosage form is retained in the stomach for at least four hours and is suitable for once daily or twice daily administration.

### Emb-3

A pharmaceutical dosage form comprising a therapeutically effective amount of at least one opioid, acetaminophen, and at least one pharmaceutically acceptable excipient wherein the said dosage form is retained in the stomach for at least four hours, wherein between on an average 10% and 30% per hour of acetaminophen initially present at 0 hours, is released between 0 and 2 hours when tested in vitro using a USP Type I apparatus in 50 mM phosphate, pH 6.8, and stirring between 50 and 150 rpm.

### Emb-4

A pharmaceutical dosage form comprising: a) a core comprising at least two release layers wherein a first release layer comprising at least one opioid with or without acetaminophen, and a second release layer comprising acetaminophen without an opioid; and b) a coat comprising the said core, wherein the dosage form is retained in the stomach for at least four hours and is suitable for once daily or twice daily administration.

### Emb-5

A method of treating a disorder by administering a pharmaceutical a dosage form comprising: a) a core comprising at least two release layers wherein a first release layer comprising at least one opioid with or without acetaminophen, and a second release layer comprising acetaminophen without an opioid; and b) a coat comprising the said core, wherein the dosage form is retained in the stomach for at least four hours and is suitable for once daily or twice daily administration.

### Emb-6

A pharmaceutical a dosage form comprising a) a compressed core comprising at least two release layers wherein a first release layer comprising at least one opioid with or without acetaminophen dispersed in a slow-release matrix; and a second release layer comprising acetaminophen with or without an opioid dispersed in a second release matrix; b) at least one permeable membrane pouch comprising the said core; c) an encapsulating coat and the said dosage form is retained in the stomach for at least four hours and is suitable for once daily or twice daily administration.

### Emb-7

A method of treating a disorder by administering a pharmaceutical a dosage form comprising a) a compressed core comprising at least two release layers wherein a first release layer comprising at least one opioid with or without acetaminophen dispersed in a slow-release matrix; and a second release layer comprising acetaminophen with or without an opioid dispersed in a second release matrix; b) at least one permeable membrane pouch comprising the said core; c) an encapsulating coat and the said dosage form is retained in the stomach for at least four hours and is suitable for once daily or twice daily administration.

### Emb-8

A pharmaceutical dosage form of Emb-1 wherein the acetaminophen is in immediate release form.

### Emb-9

A pharmaceutical dosage form of Emb-1 wherein the acetaminophen is in slow release form.

### Emb-10

A pharmaceutical dosage form comprising: a) a core comprising at least two release layers wherein a first release layer comprising at least one opioid with or without acetaminophen, and a second release layer comprising acetaminophen without an opioid; and b) a coat comprising the said core, wherein between on an average 10% and 25% per hour of opioid initially present at 0 hours, is released between 0 and 2 hours when tested in vitro using a USP Type I apparatus in 50 mM phosphate, pH 6.8, and stirring between 50 and 150 rpm.

### Emb-11

A pharmaceutical dosage form comprising: a) a core comprising at least two release layers wherein a first release layer comprising at least one opioid with or without acetaminophen, and a second release layer comprising acetaminophen without an opioid; and b) a coat comprising the said core, wherein when tested in vitro using a USP Type I apparatus in 50 mM phosphate, pH 6.8, and stirring between 50 and 150 rpm,

Between 10% and 25% of opioid released between 0 and about 2 hours of measurement,

Between about 30% and 60% of opioid released between 2 and about 7 hours of the measurement,

Between about 50% and 75% of opioid released between 7 and about 12 hours of measurement, and

Between about 75% and 100% of opioid released after about 20 hours of measurement.

### Emb-12

A pharmaceutical dosage form of Emb-1 wherein at least one layer comprises a physical mixture of polyvinyl acetate and polyvinylpyrrolidone.

### Emb-13

A pharmaceutical dosage form of Emb-1 wherein at least one layer comprises a physical mixture of polyvinyl acetate, polyvinylpyrrolidone, a binder, opioid; and wherein: the ratio of the first release layer/second release layer (w/w) is between from about 1.0 and to about 0.1.

### Emb-14

A pharmaceutical dosage form of Emb-1 for use for a period of every four hours, or every six hours, every eight hours, every twelve hours or every twenty-four hours.

### Emb-15

A pharmaceutical dosage form of Emb-1, wherein either the first release layer or the second release layer or both prepared by compression.

### Emb-16

A process of preparing a pharmaceutical dosage form comprising a) a core comprising at least two release layers wherein a first release layer comprising at least one opioid with or without acetaminophen, and a second release layer comprising acetaminophen without an opioid; and b) a coat comprising the said core, wherein the second release layer is compressed over a separately prepared said first release layer.

### Emb-17

A pharmaceutical dosage form of Emb 1-16 wherein the number of release layers is at least two and wherein the order of layers is immaterial.

### Emb-18

A pharmaceutical dosage form of Emb 1-17 wherein the dosage form comprises from about 1 to about 800 mg of opioid, and from about 1 mg to about 1000 mg of acetaminophen.

### Emb-19

A pharmaceutical dosage form of Emb 1-18, when administered to a patient in need thereof, provides a mean time to maximum plasma concentration (Tₘₐₓ) of opioid ranging from about four to about sixteen hours and the said dosage form is suitable once daily or twice daily administration.

### Emb-20

A pharmaceutical dosage form of Emb 1-19 wherein the said opioid is alfentanil, axomadol, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, faxeladol, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papavereturn, pentazocine, phenadoxone, phenomorphan, phenazocine, phenoperidine, piminodine, piritramide, propheptazine, promedol, properidine, propoxyphene, sufentanil, tilidine, tapentadol, and tramadol and the dosage form is retained in stomach for at least four hours and is suitable for once daily or twice daily administration.

### Emb-21

A pharmaceutical dosage form comprising an Opioid, acetaminophen and an opioid antagonist and at least one pharmaceutically acceptable excipient for treating pain and pain related disorders.

### Emb-22

A pharmaceutical dosage form comprising a therapeutically effective amount of at least one opioid, acetaminophen, and at least one pharmaceutically acceptable excipient wherein the said dosage form is retained in the stomach for at least four hours, wherein between on an average 10% and 30% per hour of opioid initially present at 0 hours, is released between 0 and 2 hours when tested in vitro using a USP Type I apparatus in 50 mM phosphate, pH 6.8, and stirring between 50 and 150 rpm.

What is claimed is:

1. A pharmaceutical dosage form comprising a therapeutically effective amount of at least one opioid, acetaminophen and at least one pharmaceutically acceptable excipient wherein the said dosage form is retained in the stomach for at least four hours.

2. A pharmaceutical dosage form according to claim 1, comprising a therapeutically effective amount of at least one opioid, acetaminophen, and at least one pharmaceutically acceptable excipient wherein the said dosage form is retained in the stomach for at least four hours, wherein between on an average 10% and 30% per hour of opioid initially present at 0 hours, is released between 0 and 2 hours when tested in vitro using a USP Type I apparatus in 50 mM phosphate, pH 6.8, and stirring between 50 and 150 rpm.

3. A pharmaceutical dosage form according to claim 1, comprising: a) a core comprising at least two release layers wherein a first release layer comprising at least one opioid with or without acetaminophen, and a second release layer comprising acetaminophen without an opioid; and b) a coat comprising the said core, wherein the dosage form is retained in the stomach for at least four hours and is suitable for once daily or twice daily administration.

4. A pharmaceutical a dosage form according to claim 1, comprising a) a compressed core comprising at least two release layers wherein a first release layer comprising at least one opioid with or without acetaminophen dispersed in a slow-release matrix; and a second release layer comprising acetaminophen with or without an opioid dispersed in a second release matrix; b) at least one permeable membrane pouch comprising the said core; c) an encapsulating coat and the said dosage form is retained in the stomach for at least four hours and is suitable for once daily or twice daily administration.

5. A pharmaceutical dosage form according to claim 1, wherein the acetaminophen is in immediate release form.

6. A pharmaceutical dosage form according to claim 1, wherein the acetaminophen is in slow release form.

7. A pharmaceutical dosage form according to claim 1, comprising: a) a core comprising at least two release layers wherein a first release layer comprising at least one opioid with or without acetaminophen, and a second release layer comprising acetaminophen without an opioid; and b) a coat comprising the said core, wherein between on an average 10% and 25% per hour of opioid initially present at 0 hours, is released between 0 and 2 hours when tested in vitro using a USP Type I apparatus in 50 mM phosphate, pH 6.8, and stirring between 50 and 150 rpm.

8. A pharmaceutical dosage form according to claim 1, comprising: a) a core comprising at least two release layers wherein a first release layer comprising at least one opioid with or without acetaminophen, and a second release layer comprising acetaminophen without an opioid; and b) a coat comprising the said core, wherein when tested in vitro using a USP Type I apparatus in 50 mM phosphate, pH 6.8, and stirring between 50 and 150 rpm,
Between 10% and 25% of opioid released between 0 and about 2 hours of measurement,
Between about 30% and 60% of opioid released between 2 and about 7 hours of the measurement,
Between about 50% and 75% of opioid released between 7 and about 12 hours of measurement, and
Between about 75% and 100% of opioid released after about 20 hours of measurement.

9. A pharmaceutical dosage form according to claim 1, wherein at least one layer comprises a physical mixture of polyvinyl acetate and polyvinylpyrrolidone.

10. A pharmaceutical dosage form according to claim 1, wherein at least one layer comprises a physical mixture of polyvinyl acetate, polyvinylpyrrolidone, a binder, opioid; and wherein: the ratio of the first release layer/second release layer (w/w) is between from about 1.0 and to about 0.1.

11. A pharmaceutical dosage form according to claim 1, adapted to be used for a period of every four hours, or every six hours, every eight hours, every twelve hours or every twenty-four hours.

12. A pharmaceutical dosage form according to claim 1, wherein either the first release layer or the second release layer or both prepared by compression.

13. A pharmaceutical dosage form according to claim 1, wherein the number of release layers is at least two and wherein the order of layers is immaterial.

14. A pharmaceutical dosage form according to claim 1, wherein the dosage form comprises from about 1 to about 800 mg of opioid, and from about 1 mg to about 1000 mg of acetaminophen.

15. A pharmaceutical dosage form according to claim 1, which, when administered to a patient in need thereof, provides a mean time to maximum plasma concentration (Tₘₐₓ) of opioid ranging from about four to about sixteen hours and the said dosage form is suitable once daily or twice daily administration.

16. A pharmaceutical dosage form according to claim 1, wherein the said opioid is selected from the group consisting of alfentanil, axomadol, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, faxeladol, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papavereturn, pentazocine, phenadoxone, phenomorphan, phenazocine, phenoperidine, piminodine, piritramide, propheptazine, promedol, properidine, propoxyphene, sufentanil, tilidine, tapentadol, and tramadol and the dosage form is retained in stomach for at least four hours and is suitable for once daily or twice daily administration.

17. A pharmaceutical dosage form according to claim 1, comprising an Opioid, acetaminophen and an opioid antagonist and at least one pharmaceutically acceptable excipient and adapted for treating pain and pain related disorders.

18. A process of preparing a pharmaceutical dosage form according to claim 1, comprising a) a core comprising at least two release layers wherein a first release layer comprising at least one opioid with or without acetaminophen, and a second release layer comprising acetaminophen without an opioid; and b) a coat comprising the said core, said process comprising compressing the second release layer over a separately prepared said first release layer.

19. A method of treating a disorder in a patient in need thereof, said method comprising administering an effective amount therefor of a pharmaceutical dosage form according to claim 1.

20. A method of treating a disorder according to claim 19, wherein the dosage form comprises: a) a core comprising at least two release layers wherein a first release layer comprising at least one opioid with or without acetaminophen, and a second release layer comprising acetaminophen without an opioid; and b) a coat comprising the said core, wherein the dosage form is retained in the stomach for at least four hours and is suitable for once daily or twice daily administration.

21. A method of treating a disorder according to claim 19, wherein the dosage form comprises: a) a compressed core comprising at least two release layers wherein a first release layer comprising at least one opioid with or without acetaminophen dispersed in a slow-release matrix; and a second release layer comprising acetaminophen with or without an opioid dispersed in a second release matrix; b) at least one permeable membrane pouch comprising the said core; c) an encapsulating coat and the said dosage form is retained in the stomach for at least four hours and is suitable for once daily or twice daily administration.
